SEQUENCE LISTING (18 sequences)

SEQ ID NO: 1 - DNA, 1497 bp, Artificial Sequence
  Description of Artificial Sequence/Note = Synthetic Construct
ctgcagcttgttctttaatgtcaggagactctcccttctgcttgtcctggtgggccctggggggagcggggagggaatacctaagagcaattggtagctggtacttctaatgcctcttcctcctccaacctccaagagtctgttttgggattgggttcaggaatgaaattctgcctgtgctaacctcctggggagccggtagacttgtctgttaaaaatcgcttctgcttttggagcctaaagcccggttccgaaaaacaagtggtatttaggggaaagaggggtcttcaaaggctacagtgagtcattccagccttcaaccatactacgccagcactacgttctctaaagccactctgcgctagcttgcggtgaggggaggggagaaaaggaaaggggaggggaggggaggggagggagaaaggaggtgggaaggcagagaggccggctgcgggggcgggaccgactcacaaactgttcgatttcgtttccacctcccagcgccccctcggagatccctaggagccagcctgctgggagaaccagagggtccggagcaaacctggaggctgagagggcatcagaggggaaaagactgagctagccactccagtgccatacagaagcttaagggacgcaccacgccagccccagcccagcgacagccaacgcctgttgcagagcggcggcttcgaagccgccgcccaggagctgccctttcctcttcggtgaagtttctaaaagctgcgggagactcagaggaagcaaggaaagtgtccggtaggactacggctgcctttgtcctcttcccctctacccttaccccctcctgggtcccctctccaggagctgactaggcaggctttctggccaaccctctcccctacacccccagctctgccagccagtttgcacagaggtaaactccctttggctgagagtaggggagcttgttgcacattgcaaggaaggcttttgggagcccagagactgaggagcaacagcacgcccaggagagtccctggttccaggttctcgcccctgcacctcctcctgcccgcccctcaccctgtgtgtggtgttagaaatgaaaagatgaaaaggcagctagggtttcagtagtcgaaagcaaaacaaaagctaaaagaaaacaaaaagaaaatagcccagttcttatttgcacctgcttcagtggactttgaatttggaaggcagaggatttccccttttccctcccgtcaaggtttgagcatcttttaatctgttcttcaagtatttagagacaaactgtgtaagtagcagggcagatcctgtcttgcgcgtgccttcctttactggagactttgaggttatctgggcactccccccacccaccccccctcctgcaagttttcttccccggagcttcccgcaggtgggcagctagctgcagatactacatcatcagtcaggagaactcttcagagcaagagacgaggaggcaggataagggaattc

SEQ ID NO: 2 - DNA, 600 bp, Artificial Sequence
  Description of Artificial Sequence/Note = Synthetic Construct
ctgcagcttgttctttaatgtcaggagactctcccttctgcttgtcctggtgggccctggggggagcggggagggaatacctaagagcaattggtagctggtacttctaatgcctcttcctcctccaacctccaagagtctgttttgggattgggttcaggaatgaaattctgcctgtgctaacctcctggggagccggtagacttgtctgttaaaaatcgcttctgcttttggagcctaaagcccggttccgaaaaacaagtggtatttaggggaaagaggggtcttcaaaggctacagtgagtcattccagccttcaaccatactacgccagcactacgttctctaaagccactctgcgctagcttgcggtgaggggaggggagaaaaggaaaggggaggggaggggaggggagggagaaaggaggtgggaaggcagagaggccggctgcgggggcgggaccgactcacaaactgttcgatttcgtttccacctcccagcgccccctcggagatccctaggagccagcctgctgggagaaccagagggtccggagcaaacctggaggctgagagggcatcagaggggaaaagactgag

SEQ ID NO: 3 - DNA, 359 bp, Artificial Sequence
  Description of Artificial Sequence/Note = Synthetic Construct
cccaagcgctagtgttctgttctctttttgtaatcttggaatcttttgttgctctaaatacaattaaaaatggcagaaacttgtttgttggaatacatgtgtgactcttggtttgtctctgcgtctggctttagaaatgtcatccattgtgtaaaatactggcttgttggtctgccagctaaaacttgccacagcccctgttgtgactgcaggctcaagttattgttaacaaagagccccaagaaaagctgctaatgtcctcttatcaccattgttaatttgttaaaacataaaacaatctaaaatttcagatgaatgtcatcagagttcttttcattagctctttttattggctgtct

SEQ ID NO: 4 - protein, 899 aa, Artificial Sequence
  Description of Artificial Sequence/Note = Synthetic Construct
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 5 - DNA, 2988 bp, Artificial Sequence
  Description of Artificial Sequence/Note = Synthetic Construct
gcttcccgcaggtgggcagctagctgcagatactacatcatcagtcaggagaactcttcagagcaagagacgaggaggcaggataagggaattcggtggaagctacagacaagctcaaggatggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtatcctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtgaagatttggaaaccctaatacccaaaacccaccttgttccctttccagatgtcttctgcctgttatataactctgcactacttctctgcagtgccttgggggaaattcctctactgatgtacagtcagacgtgaacaggttcctcagttctatttcctgggcttctcct

SEQ ID NO: 6 - protein, 899 aa, Artificial Sequence; identical to SEQ ID NO: 4
  Description of Artificial Sequence/Note = Synthetic Construct

SEQ ID NO: 7 - DNA, 2988 bp, Artificial Sequence; identical to SEQ ID NO: 5
  Description of Artificial Sequence/Note = Synthetic Construct

SEQ ID NO: 8 - protein, 899 aa, Artificial Sequence; identical to SEQ ID NO: 4
  Description of Artificial Sequence/Note = Synthetic Construct

SEQ ID NO: 9 - DNA, 2700 bp, Artificial Sequence
  Description of Artificial Sequence/Note = Synthetic Construct
atggaggtgcagttagggctgggaagggtctacccacggcccccatccaagacctatcgaggagcgttccagaatctgttccagagcgtgcgcgaagcgatccagaacccgggccccaggcaccctgaggccgctaacatagcacctcccggcgcctgtttacagcagaggcaggagactagcccccggcggcggcggcggcagcagcacactgaggatggttctcctcaagcccacatcagaggccccacaggctacctggccctggaggaggaacagcagccttcacagcagcaggcagcctccgagggccaccctgagagcagctgcctccccgagcctggggcggccaccgctcctggcaaggggctgccgcagcagccaccagctcctccagatcaggatgactcagctgccccatccacgttgtccctgctgggccccactttcccaggcttaagcagctgctccgccgacattaaagacattttgaacgaggccggcaccatgcaacttcttcagcagcagcaacaacagcagcagcaccaacagcagcaccaacagcaccaacagcagcaggaggtaatctccgaaggcagcagcgcaagagccagggaggccacgggggctccctcttcctccaaggatagttacctagggggcaattcaaccatatctgacagtgccaaggagttgtgtaaagcagtgtctgtgtccatgggattgggtgtggaagcattggaacatctgagtccaggggaacagcttcggggagactgcatgtacgcgtcgctcctgggaggtccacccgcggtgcgtcccactccttgtgcgccgctgcccgaatgcaaaggtcttcccctggacgaaggcccaggcaaaagcactgaagagactgctgagtattcctctttcaagggaggttacgccaaaggattggaaggtgagagcttggggtgctctggcagcagtgaagcaggtagctctgggacacttgagatcccgtcctctctgtctctgtataaatctggagcactagacgaggcagcagcataccagaatcgcgactactacaactttccgctggctctgtccgggccgccgcaccccccgccccctacccatccacacgcccgtatcaagctggagaacccattggactacggcagcgcctgggctgcggcggcagcgcaatgccgctatggggacttgggtagtctacatggagggagtgtagccgggcccagcactggatcgcccccagccaccacctcttcttcctggcatactctcttcacagctgaagaaggccaattatatgggccaggaggcgggggcggcagcagcagcccaagcgatgccgggcctgtagccccctatggctacactcggccccctcaggggctgacaagccaggagagtgactactctgcctccgaagtgtggtaccctggtggagttgtgaacagagtaccctatcccagtcccaattgtgtcaaaagtgaaatgggaccttggatggagaactactccggaccttatggggacatgcgtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaagggaaacagaagtatctatgtgccagcagaaacgattgtaccattgataaatttcggaggaaaaattgcccatcttgtcgtctccggaaatgttatgaagcagggatgactctgggagctcgtaagctgaagaaacttggaaatctaaaactacaggaggaaggagaaaactccaatgctggcagccccactgaggacccatcccagaagatgactgtatcacacattgaaggctatgaatgtcagcctatctttcttaacgtcctggaagccattgagccaggagtggtgtgtgccggacatgacaacaaccaaccagattcctttgctgccttgttatctagcctcaatgagcttggagagaggcagcttgtgcatgtggtcaagtgggccaaggccttgcctggcttccgcaacttgcatgtggatgaccagatggcggtcattcagtattcctggatgggactgatggtatttgccatgggttggcggtccttcactaatgtcaactccaggatgctctactttgcacctgacttggttttcaatgagtaccgcatgcacaagtctcggatgtacagccagtgtgtgaggatgaggcacctgtctcaagagtttggatggctccaaataaccccccaggaattcctgtgcatgaaagcactgctgctcttcagcattattccagtggatgggctgaaaaatcaaaaattctttgatgaacttcgaatgaactacatcaaggaactcgatcgcatcattgcatgcaaaagaaagaatcccacatcctgctcaaggcgcttctaccagctcaccaagctcctggattctgtgcagcctattgcaagagagctgcatcagttcacttttgacctgctaatcaagtcccatatggtgagcgtggactttcctgaaatgatggcagagatcatctctgtgcaagtgcccaagatcctttctgggaaagtcaagcccatctatttccacacacagtga

SEQ ID NO: 10 - DNA, 2217 bp, Artificial Sequence
  Description of Artificial Sequence/Note = Synthetic Construct
cacccccccaatcccctacccacccactccccctttttggccctggcgttcccctgtactggggcatataaagtttgcaagtccaatgggcctctctctttgccatgatggccgactaggccatcttttgatacatatgcagctaaagacaagagctcccgggtactggttagttcatattgttgttccacctatagggttgcagttccctttagctccttgggtaatttctctagctcctccattaggggccgtgtgacccatccaatagctgactgtgatcatccacttctgtgtttgctaggccccgacatagtctcacaagagagagctataactgggtcctttcagcgaaatcttgctagtgtatgcaatggtgtcagcatttggaagctgattatgggatggatccctgcatatggcatctattacatttttgttacagaacagggaaagggacactgagagactcaagaagaaagaaaaggaattaatacaaaagaacagtgaaagctggtatgataatactaatttatcctttacttgtatattaatatcaagagtaactcatacatctgatttatgttgtcagagcaataactcagtactactggtagcaatattgntgtttttacagggtaagactctaggctccaagagctaaaatatataaaattcttctggtatttgataaggctgatcataggcctctctctggaagaagtaagatagagttatgttcatgccatttaatgactgtatatgtcgtcattaatgcatcacattaagttgataccttaacctctgcttaacttccttctcttacaaatgcagagctcatgagattggctattccctcagaacctgtttaattccttggcaggattcaaagtgtccataggaaaccttacaaacactctgtccagagaaggtctcaaaagagttcagctttacactgattcactcgagcaatccatagaatagtcacttggatgtatgtacagtttctcagaagaccgtagaattctgatcgatgtctgccatccactgacatatgttgctttgttctctctctgtctctgtgtgtgtcttttcagtttggacagtaccagggaccatgttttacccatcgactattactttccaccccagaagacctgcctgatctgtggagatgaagcttctggctgtcactacggagctctcacttgtggcagctgcaaggtcttcttcaaaagagccgctgaaggtaaaaagtcttacctacttcctgatattttccccttctcttttgcctagcagagaatgacagtgaccttccagggcattctgataatcccagagactgagtcattagcaagggccctctcacagtacatgtaagatcaaagaagcccatggttatatttgctgagctgtcttggctgccctggttgtacaagcaatgatggtgatgtaggtggtcccagctggtgcttggtggctcccaggactggaagcaaaattaatgatttgaaaaattaaatttccttcctgcttgttttcaactctgcttcctagtgaggaaaagaaaacttgtccttattagagaggttagaagtggagaaaccccaactgagtatacaggctgttttctgtagagaatatgagactgttccttagcaaaagcttcctggctttaaccccagaaaaggaagtgttctcactgttcagcagaccatcagtgtctgcacctgctccctcctgcttgctgcctctttgggacctctctttgcaataagggactccaangcangaaaaaaactcagagagaagcatcagaggactgctttcagggcatgacagttggttcaagaatcccaacgtaacttgcattttgtatccagctaagtgggatggagcctttacttgttatctgcactaattatgatgtttctaacctacatcatctagcagaaacacccactccaggcctttactgtagtcttagtgatccctcccttcttaatcacagggtgggggtgggagcttaaacctttattcatacactctactaccatccctcagtctggtactcctttctcaaagagtcactggaaagctgcccctacatggtctactgtggctgcagactcagttttaaagattcctttgcaactctgccctggtctctggcttcccaccaagggggancttccggccagggaggttttcctt

SEQ ID NO: 11 - DNA, 20 bp, Artificial Sequence
  Description of Artificial Sequence/Note = Synthetic Construct
gttgataccttaacctctgc

SEQ ID NO: 12 - DNA, 20 bp, Artificial Sequence
  Description of Artificial Sequence/Note = Synthetic Construct
cctacatgtactgtgagagg

SEQ ID NO: 13 - DNA, 19 bp, Artificial Sequence
  Description of Artificial Sequence/Note = Synthetic Construct
ttcagcggctcttttgaag

SEQ ID NO: 14 - DNA, 28 bp, Artificial Sequence
  Description of Artificial Sequence/Note = Synthetic Construct
tctagaactgtcctgaccatgtgtaatt

SEQ ID NO: 15 - DNA, 68 bp, Artificial Sequence
  Description of Artificial Sequence/Note = Synthetic Construct
atcactcgagataacttcgtataatgtatgctatacgaagttatggtaccctcgagctttccatagaa

SEQ ID NO: 16 - DNA, 66 bp, Artificial Sequence
  Description of Artificial Sequence/Note = Synthetic Construct
tctagaaagcttgatatcgtcgaataacttcgtataatgtatgctatacgagttatgtcgagcccc

SEQ ID NO: 17 - DNA, 72 bp, Artificial Sequence
  Description of Artificial Sequence/Note = Synthetic Construct
gtcgataacttcgtataatgtatgctatactaagttatgtcgacctaggaattcctctcacagtacatgtag

SEQ ID NO: 18 - DNA, 33 bp, Artificial Sequence
  Description of Artificial Sequence/Note = Synthetic Construct
tacagtttctcagaagaccgtagaattcagatc

(19) United States
(12) Patent Application Publication
(10) Pub. No.: US 2004/0261139 A1
(43) Pub. Date: Dec. 23, 2004

(54) Androgen receptor knock-out transgenic animals

(76) Inventors: Chawnshang Chang, Pittsford, NY (US); Shuyuan Yeh, Rochester, NY (US)

Correspondence Address: NEEDLE & ROSENBERG, P.C., SUITE 1000, 999 PEACHTREE STREET, ATLANTA, GA, 30309-3915, US

(21) Appl. No.: 10/484,950

(22) PCT Filed: Jul. 29, 2002
(86) PCT No.: PCT/US02/24234

Related U.S. Application Data
(60) Provisional application No. 60/381,309, filed on May 17, 2002.
(60) Provisional application No. 60/308,356, filed on Jul. 27, 2001.

(51) Int. Cl. A01K 67/027; C12N 5/06

(52) U.S. Cl. 800/18; 435/354

(57) ABSTRACT

Disclosed are compositions and methods for disrupting an androgen receptor.

### II. CROSS REFERENCE TO RELATED APPLICATIONS

This application claims benefit of U.S. Provisional Application Ser. No. 60/381309, filed May 17, 2002, and U.S. Provisional Application Ser. No. 60/308356, filed Jul. 27, 2001, both of which are hereby incorporated herein by reference in their entirety.

### I. ACKNOWLEDGMENTS

This invention was made with government support under Grants CA55639 and CA71570. The government has certain rights in the invention.

### III. BACKGROUND OF THE INVENTION

Androgens have been extensively characterized as mediating developmental and physiological responses in men and are implicated in a number of male pathological conditions, most notably prostate cancer. However, androgen action may also play a contributory or inhibitory role in cancer progression in women. Clinically and in animal studies, androgens have an inhibitory effect on breast cancer growth (1-4). Conversely, elevated androgen levels may contribute to the risk of ovarian cancer (5). A combination of androgens and estrogens may be important for the prevention or treatment of post-menopausal osteoporosis (6).

It has been recognized that estrogen deficiency plays a major role in post-menopausal alterations in bone metabolisms contributing to osteoporosis. However, androgens may also have an important role in the maintenance of bone density. AR and the estrogen receptor (ER) are expressed in bone cells including osteoblasts and osteoclasts, the cell type that predominantly mediate bone formation and bone resorption, respectively (10,11). Clinically, a positive correlation is observed between bone mass and serum androgen levels in both men and women (12-14) and androgen treatment can increase bone mass in hypogonadal men (15). The decreased ovarian function that occurs with menopause results in an approximately 80% reduction in estrogens and an approximately 50% reduction in androgens (16). While estrogen treatment of post-menopausal osteoporosis prevents bone loss, combined treatment of estrogen and androgen has been found to have a more positive effect on bone density, possibly through the ability of androgens to stimulate bone formation (17-19).

Ovarian hormones have long been recognized as playing an important role in breast cancer development and as stimulators of breast cancer growth. In contrast, the bulk of experimental and clinical evidence implicates androgens in the inhibition of breast cancer proliferation. In a rat model of breast cancer development, supraphysiological doses of testosterone have been found to shorten the latency of mammary tumor formation (20). However, in this system it is unclear how much of the administered testosterone is converted to estrogen in the mammary gland. The human derived breast cancer cell line MDA-MB-453 has been reported to proliferate in response to androgens (21), while MCF-7 cells have alternatively been reported to proliferate in response to the nonaromatizable androgen DHT (21), or to show no proliferative response to DHT (22). However, DHT inhibits estrogen-induced proliferation of the AR positive breast tumor derived cell lines MFM-223 (22), T47-D (21), ZR-75-1 (23), and MCF-7 cells stably transfected with AR (24). These anti-proliferative effects are blocked by the addition of antiandrogens (e.g. hydroxyflutamide or casodex) suggesting that the inhibition of proliferation is modulated by AR. In a nude mouse xenograft model of breast cancer, estradiol stimulated growth of injected ZR-75-1 cells was inhibited by physiological levels of DHT (4). Additionally, DHT inhibited estradiol enhanced tumor growth in ovarectomized rats harboring DMBA induced mammary tumors (3). This antiproliferative effect was reversed in the presence of the antiandrogen flutamide. Clinically, androgens or androgenic compounds such as testosterone propionate (1), calusterone (25,26), and fluoxymesterone (2) have been found to be effective adjuvant therapies for breast cancer in both pre- and postmenopausal patients. However, the negative side effects of androgen therapy in women have limited its therapeutic use. The AR is expressed in 50-85% of breast tumors (27,28).

Disclosed herein are compositions and methods, including vectors, for making androgen receptor knockout mice, as well as the mice themselves. Also disclosed are androgen receptor knockout mice, both male and female, which can be used to study the role of androgen rceptor in cancer as well as reagents to test therapeutics targeting cancer. The disclosed mice are inducible knockouts, meaning that they were constructed to so that the androgen receptor can be knockout through production of a recombinase, such as Cre recombinase. Therefore, the disclosed mice can be crossed with any strain of cre producing mouse under any promoter system, to generate any tissue specific knockout of androgen receptor. Also disclosed are cell lines which have had the androgen receptor knocked out.

### IV. SUMMARY OF THE INVENTION

In accordance with the purposes of this invention, as embodied and broadly described herein, this invention, in one aspect, relates to compositions and methods related to androgen receptor.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### V. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the homologous recombination and disruption of the AR loci in MCF-7 breast cancer cells. A, Schematic diagram of the AR targeting vector and the predicted product of homologous recombination with the AR locus. The AR targeting vector was constructed in the pGEM-T easy vector (Promega) and contains 1.1 kb of the AR 5′ UTR, 0.1 kb of the AR exon1, a promoterless neomycin cassette (1.2 kb), and 6.2 kb of the AR intron 1. AR sequences were obtained by PCR from LNCAP cells. Prior to transfection into MCF-7 cells, the construct was verified by sequencing. B. MCF-7 cells were transfected with the AR targeting vector using Superfect (Qiagen) and transfectants selected in 400 μg/ml G418. The genotype of the clones surviving selection were screened by Southern blot. Homologous integrants were identified by Southern blot using an XbaI digestion and a probe to the AR 5′ UTR as depicted in the upper panel. The untargeted locus gives an XbaI fragment of 9.0 kb and the targeted locus produces a 3.458 kb band. After this first round of targeting, only heterologous clones were isolated. One of these clones was re-transfected with the targeting vector and selected in media containing a higher concentration of G418 (1.25 mg/.ml). The surviving clones were again screened by Southern blot for the presence of homolgous integrants. The Southern blot shown depicts the isolation of an MCF-7 clone lacking an intact AR locus (MCF-ARKO, −/−) and for comparison, clone heterozygous for the targeted locus (+/−) and the parental cell line (+/+).

FIG. 2 shows a construction of the flox AR targeting vector. The PKI vector is modified from the pBluescript plasmid. It contains a T7promoter at 3′ end T3 promoter at 5′ end, two multiple cloning sites (MCS), two lox sites ( [symbol] ), a positive Neo selective marker (PKG-Neo^{r}) and a negative thymidine kinase selective marker (MCT-TK). For the cloning, the XhoI site at 5′ end MCS was first destroyed. A 3 kb intron 2 fragment was introduced into 3′ EcoR1 cloning site (R1). A fragment containing intron1, exon2 and a small fragment of intron2 was inserted into the 5′ XbaI site (X). A lox sequence plus an artificial KpnI cutting site were finally inserted to the XhoI site shortly 5′ to exon 2. The sequence of the targeting construct was verified by DNA sequencing. For electroporation into ES cells, the plasmid can be linearized at a unique NotI site.

FIG. 3 shows screening of the extracted DNA to distinguish wild type AR from floxAR: (A)AR fragment and the flanking region: The restriction fragment of KpnI in wild type is 9 kb. There are three lox sites and an artificial KpnI restriction site in floxAR fragment. The KpnI restriction will result in one 7⁻kb and one5⁻kb fragments in flox AR. By using the 3′ end sequence as the probe (Pb), The southern blot hybridization would display a 9 kb fragment in wild type ES cell clones and a strong 7 kb fragment (ES cells) plus a weak 9 kb fragment (STO cells) in specific recombinated ES cell clones. Using the Pm1, Pm2 and PmNeo as the primers, the multiplex PCR would generate a 400⁺bp product in wild type cells and a 600⁺bp product in recombinated cells. (B) Southern blot screening of the embryonic stem cells transfected with floxAR: #1 and #7 clones are recombinated specifically. It displayed a strong signal at 7 kb position (ES cells) and a weak signal at 9 kb position (STO cells). #2 to #6 are the wild type that displayed signal only at 9 kb position. (C) Southern blot screening of the flox AR clone transfected with pCMV plasmid. The pCMV-cre restricted the sequence between two lox sites and would generate 4 types. #1 is the one without restriction (7 kb), #2 is typeI restriction (5 kb), #3 is typeII (11 kb) and #4 is the type III restriction (9 kb).

(D) Multiplex polymerase chain reaction for genotype screening of the mice:The chimera mice displayed a 400⁺bp band and a 600⁺bp band (lane1). The wild type display only 400⁺bp band (lane2).

FIG. 4 shows the generation of female mice lacking AR. Only the desired genotypes are shown in this figure. Chimeric mice for flox AR (fAR) containing cells (B6/129 chimeras were mated to C57B1/6J females to generate the flox AR mice. These animals were crossed to transgenic mice carrying the ACTB-Cre transgene. Finally, flox AR males were crossed to heterozygous flox AR, ACTB-Cre females to generate females that are homozygous for flox AR and carry the ACTB-Cre transgene. This genetic combination resulted in Cre expression prior to the blastocyst stage of embryonic development causing recombination between the lox sites flanking exon 2, resulting in its excision and disruption of the AR coding sequence as described in the text. To identify these female mice, tail biopsies were taken and screened for recombined AR locus by Southern blot or PCR as described in the preliminary results (C-2). Mice were also screened for presence of the Cre transgene and Sry by PCR The chimera founder is B6/129 mosaic strain. Thus, the mating of the founder with the B6 female will create female mice heterozygous with flox AR. The following F2 generation will generate floxAR, male mice. The mating between the heterozygous floxAR female and the homozygous FVB/N-TgN ALTB cre male that carry cre-recombinase under β-actin promoter will create a female heterozygous floxAr carrying the cre recombinase. The mating between the floxAR male and the heterozygous female carrying the cre recombinase will generate the homozygous floxAR cre+ female mice.

FIG. 5 shows the genotyping of AR KO mice. As shown in FIG. 4, we have applied primer “select” and “2-9” to identify wt and AR KO male mice in our study. (A) Schematic presentation of the DNA construct and primer location in exon 2 area of wt, KO and Floxed AR gene and the list of the sizes of PCR product amplified by designed primer pairs. (B) The Identification of wt and KO AR mice, Using select and 2-9, we have amplified a DNA fragment with 580 bp which represents wt AR, and with 238 bp which represent KO of AR exon 2. The expression of Cre and internal control IL2 were confirmed by PCR on the bottom panel.

FIG. 6 shows the potentiation of AR transactivation by BRCA1. A. BRCA1, but not p53, potentiates wild type AR transactivation in prostate cancer cells, but not transactivation of an AR DNA binding domain mutant AR. In each 60 mm plate of DU145 cells, 1 μg of pSG5-AR, 3 μg of MMTV-CAT, in the presence or absence of 4.5 μg of pCR3-BRCA1 or p53 as indicated. Cells were transfected by calcium phosphate precipitation and treated with DHT for 24 hrs before harvesting. B. BRCA1 can potentiate AR transcription in LNCaP cells in the presence of androgen but does not influence the protein level of the endogenous AR. In each 35-mm dish of LNCaP cells, 0.5 μg of PSA-Luc and 1.0 μg of pCR3 or pCR3-BRCA1 were transfected using Superfect (Qiagen). C. BRCA1 can potentiate AR transactivation in T47D and MCF-7 cells. Cells were transfected as in B.

FIG. 7 shows that AR coregulators can cooperate with BRCA1 to enhance AR transactivation. DU145 cells were transfected with 3 μg of MMTV-CAT, 1 μg of pSG5-AR, and 3 μg alone or in combination of CBP, ARA70N, ARA55, or BRCA1 in the presence or absence of 1 nM DHT as indicated.

FIG. 8 shows that BRCA1 enhances AR mediated transcription of the endogenous p21(WAF1/CIP1) gene in MCF-7 and PC-3(AR2) cells. A. MCF-7 cells were transfected using Superfect (Qiagen) with 2 μg of pSG5-AR with or without 4 μg of BRCA1, as indicated. Cells were cultured in the presence of 10 nM DHT or vehicle, as indicated. B. PC-3(AR2) cells were transfected under the same conditions as the MCF-7 cells in A. except that AR was not transfected. C. PC-3(AR2) cells transfected with BRCA1 were cultured in the presence of vehicle, 10 nM DHT, or 1 μM hydroxyflutamide (HF).

FIG. 9 shows the effect of ARA70 on the E2-mediated AR transcriptional activity. Effects of E2, DES, estrone, estriol, 17-E2, Tam, ICI, and DHT on the transcriptional activity of AR in the presence or absence of ARA70 in DUI45 cells. After transfection, the cells were treated with serial concentrations of E2, DES, estrone (E1), estriol (E3), 17α-E2, tamoxifen (58), ICI 182, 780 (ICI), and DHT (10⁻¹⁰ M: lanes 1, 6, 11, 16, 21, 26, 31, 36; 10⁻⁹ M: lanes 2, 7, 12, 17, 22, 27, 32, 37; 10⁻⁸ M: lanes 3, 8, 13, 18, 23, 28, 33, 38; 10⁻⁷ M: lanes 4, 9, 14, 19, 24, 29, 34, 39; 10⁻⁶ M: lanes 5, 10, 15, 20, 25, 30, 35, 40). The DHT treatments were taken as the positive control. Data represents an average of three independent experiments. The variance is ±15%

FIG. 10 shows the junction sequences for the vector shown in FIG. 2. The sequence 5′-TCTAGAACTGTCCTGACCATGTGTAATT-3′ is the 5′ arm of intron 1 of the mouse androgen receptor gene which also contains an xba1 restriction site on the 5′ end. The sequence 5′-ATCACTCGAGATAACTTCGTATAATGTATGCTATACGAAGTTATGGTACCCTCGAG CTTTCCATAGAA-3′ is the 3′ end of intron 1 containing a loxP recombination site flanked by xho1 restriction sites on either side of the loxP site all contained within intron 1. The 5′ end of intron 2 has the sequence 5′-TCTAGAAAGCTTGATATCGTCGAATAACTTCGTATAATGTATGCTATACGAGTTAT GTCGAGCCCC-3′. This sequence describes the 5′ end of intron 2 of the androgen receptor gene which contains a xba1 restriction site at the 5′ end and an additional loxP recombination site as well as a neo cassette reporter gene on the 3′ end. 5′-GTCGATAACTTCGTATAATGTATGCTATACTAAGTTATGTCGACCTAGGAATTCCT CTCACAGTACATGTAG-3′ is the 3′ end of the neo cassette located within intron 2 and flanked by a loxP site on the 5′ end. Like the 5′ end, the 3′ end of the neo cassette is also flanked by a loxP recombination site. The sequence further describes more of intron 2 of the androgen receptor gene on the 3′ end of the sequence. The 3′ end of intron 2 of the androgen receptor is disclosed as 5′-TACAGTTTCTCAGAAGACCGTAGAATTCAGATC-3.′ It is recognized that one of ordinary skill in the art will recognize these sequences as describing in detail the location of the loxP sites of recombination within introns 1 and 2 of the androgen receptor as well as the location of the neo cassette in intron 2. Furthermore, it is understood that an artisan will recognize the disclosed sequences as disclosing the androgen receptor exon 1 and related introns 1 and 2, and know how to use the disclosed nucleotides to find the remaining portions of the sequence through standard recombinate biotechnology. Additionally, the disclosure of the preceding sequences will indicate to an artisan the methods and reagents used by the inventor to assemble the construct containing intron 1 and intron 2 of the androgen receptor as well as the three loxP recombination sites and the neo cassette reporter gene.

### VI. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The abbreviations used are: AR, androgen receptor; ARKO, androgen receptor knock-out; mtAR, mutant AR; ARA, androgen receptor associated protein; DHT, 5α-dihydrotestosterone; HF, hydroxyflutamide; PSA, prostate specific antigen; MMTV, mouse mammary tumor virus; CAT, chloramphenicol acetyltransferase; LUC, luciferase; DMBA, dimethylbemz(a)anthracene; WAP, whey acidic protein, LH-RH—Leutinizing hormone-releasing hormone, BPH—Benign prostatic hyperplasia, DES—diethylstilbesterol, and GnRH—Gonadotropic releasing hormone.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

B. Compositions and Methods

1. Compositions and Methods for Disrupting an AR Loci

The Cre-lox system has been successfully used herein to generate androgen receptor knockout mice (ARKO). This principle has been successfully applied for tissue-specific transgene expression (Orban PC, 1992), for site specific gene targeting (Gu, 1994) and for exchange of gene sequence by the “knock-in” method (Hank M, 1995). Disclosed herein, the system has been applied to avoid the infertility problem of male carriers of an androgen receptor knockout. This strategy has been used to generate a knock-out model for those genes that are located in X or Y chromosomes and are critical in fertility.

Disclosed are methods of generating a cell line wherein the AR loci has been disrupted. For example, the AR loci can be disrupted by, for example, disrupting one of the exons, such that a stop codon terminates translation of the AR peptide early or where the exon is completely taken out. The AR loci would include any exon or intron associated with the AR gene on the X chromosome.

The AR gene is considered any sequence associated with the AR locus. Thus, it would at least include the chromosomal nucleic acid contained within any organism that expresses an AR, such as, the introns, exons, 5′ upstream sequence involved with the AR coding and non-coding sequence, and 3′ downstream sequence involved with the AR coding and non coding sequence.

Also disclosed are methods wherein the cell line or cells is a breast cancer cell line, such as the cell line MCF-7, ZR-75-1, or T47-D or breast cancer cells.

Also disclosed are methods, wherein the cell line or cells is an ovarian cancer cell line wherein the AR loci has been disrupted, such as, OVCAR-3, ES-2, SKOV-3 or ovarian cancer cells.

Also disclosed are methods, wherein the cell line or cells is a prostate cancer cell line or prostate cancer cells, CNGP cells or cell lines, or muscle cells or cell lines, bone cells or cell lines, brain cells or cell lines.

A disrupted AR loci can be any AR loci that does not produce a native AR protein. A disrupted AR loci would also include any AR loci wherein the nucleic acid of the natural AR gene, including exons and introns has been altered. Typically the altering of the AR gene will cause a disruption in AR function, by for example, preventing DNA binding in the AR gene product or ligand binding in the AR gene product or transactivating activity in the AR gene product. The disrupted AR loci can be made using any known technique, including homologous recombination techniques. The disrupted loci can be an alteration of any exon to produce a non-functional AR protein. Furthermore, disclosed are constructs and methods to mutate any exon in the AR through homologous recombination via the surrounding introns. For example, Exon 1 can be floxed through addition of a lox site in sequence that will homologously recombine with Intron 1 and inron 2. Likewise lox sites could be inserted into sequence which would homologously recombine with intron 2 and intron 3 for Exon 2, intron 3 and intron 4 for exon 3, intron 4and intron 5 for exon 4, intron 5 and intron 6 for exon 5, and so forth for each exon which are considered disclosed herein.

The disrupted AR loci can be in any cell that contains an AR loci, such as an embryonic stem cell, an embryonic germ cell, a breast cell, a breast cancer cell, an ovary cell, an ovary cancer cell, and any cell line of cells that contain AR genes which are expressed, such as prostate cells, testis, bone, brain, neural, and muscle.

Disclosed are cells comprising a disrupted AR loci, and the cells could be breast cancer cells or breast cancer cell lines, such as, MCF-7, ZR-75-1, or T47-D, or other cells, such as an embryonic stem cell, an embryonic germ cell, a breast cell, a breast cancer cell, an ovary cell, an ovary cancer cell, and any cell line of cells that contain AR genes which are expressed, such as prostate cells, testis, bone, brain, neural, and muscle.

Also disclosed are cells comprising a disrupted AR loci wherein the cells are a ovarian cancer cell or ovarian cancer cell line, such as, OVCAR-3, ES-2, SKOV-3 or other cancer cells that contain an expressed AR gene.

Disclosed are methods of determining the effect of steroids on AR using an AR disrupted cell line, comprising administering a steroid to a any of the cells or cell lines disclosed herein containing a disrupted AR.

Disclosed are methods of generating an animal wherein the AR loci has been disrupted.

Disclosed are methods of generating an animal wherein the AR loci has been disrupted and wherein the disruption is inducible.

Disclosed are methods of generating an animal wherein the AR loci has been disrupted a) wherein the disruption is inducible and b) wherein the inducible gene is flanked by sites which can be acted upon by a recombinase, such as loxP sites.

Disclosed are methods of generating an animal wherein the AR loci has been disrupted a) wherein the disruption is inducible, b) wherein sequence associated with the AR loci is flanked by sites which can be acted upon a recombinase, such as loxP sites, and c) wherein the sites can be cleaved by a recombinase, such as cre recombinase, under the control of an inducible promoter or a constitutive promoter, such as, the CMV promoter.

Also disclosed are methods wherein the cre recombinase is under the control of the EIIA promoter, a promoter specific for breast tissue, such as the WAP promoter, a promoter specific for ovarian tissue, such as the ACTB promoter, a promoter specific for bone tissue. Any tissues specific promoter can be used. Promoters specific for prostate, testis, and neural are also disclosed.

Disclosed are inducible expression systems to generate mice without a functional androgen receptor. It is understood that many inducible expression systems exist in the art and may be used as disclosed herein. Inducible expression systems can include, but are not limited to the Cre-lox system, Flp recombinase, and tetracycline responsive promoters. The Cre recombinase system which when used will execute a site-specific recombination event at loxP sites. A segment of DNA that is flanked by the loxP sites, floxed, is excised from the transcript. To create null mice using the Cre-lox system, two types of transgenic mice are created. The first is a mouse transgenic for Cre recombinase under control of a known inducible and/or tissue-specific promoter. The second is a mouse that contains the floxed gene. These two transgenic mouse strains are then crossed to create one strain comprising both mutations. Disclosed are constructs and mice that place the androgen receptor (AR) gene in the floxed position such that upon recombination an AR null mutation is created. Control of the recombination event, via the Cre Recombinase, can be constitutive or inducible, as well as ubiquitous or tissue specific, depending on the promoter used to control Cre expression. Disclosed is a constitutive system in which the Cre recombinase is expressed from β-actin promoter. Other inducible expression systems exist and can be used as disclosed herein. Disclosed herein, a non-tissue specific promoter, β-actin, is used in the form of the FVB/N-TgN(ACTB-Cre)2Mrt (stock # 003376) mice (Jackson Laboratory, Bar Harbor, Me.). However, the CMV promoter and adenovirus EIIα promoter, for example, are also examples of ubiquitous promoters and can be substituted for β-actin to achieve the same result. Also disclosed are constructs and their use comprising the WAP promoter for the establishment of an inducible AR null mutation. Herein, B6129-TgN(WAPCre)11738Mam (stock # 003552) (Jackson Laboratory, Bar Harbor, Me.) mice are used to establish tissue-specific Cre recombinase expression, with Cre under the control of WAP. It is understood that other expression systems may be substituted for the Cre expression system disclosed herein. It is anticipated that variations in the expression system used can result in a need to change other components of the recombination event, for example, the promoter. Commercially available mice (Jackson Laboratory, Bar Harbor, Me.) that utilize the cre-lox inducible expression system include at least 129-TgN(PRM-Cre)58Og (stock # 003328), 129.Cg-Foxg1^{tm1(Cre)Skm} (stock # 004337), 129S6-Tg(Prnp-GFP/Cre) 1 Blw (stock # 003960), B6.129-Tg(Pcp2-Cre)2Mpin (stock # 004146), B6.129S4-Meox2^{CreSor} (stock # 003755), B6.Cg(D2)-TgN(xstpxLacZ)32And (stock # 002982), B6.Cg(SJL)-TgN(NesCre)1Kln (stock # 003771), B6.Cg-Tg(Rbp3-Cre)528Jxm (stock # 003967), B6.Cg-Tg(Syn1-Cre)671Jxm (stock # 003966), B6.Cg-Tg(Tek-Cre)12F1v (stock # 004128), B6.Cg-TgN(LckCre)548Jxm (stock # 003802), B6.FVB-TgN(EIIa-Cre)C5379Lmgd (stock # 003724), B6129-TgN(MMTV-Cre)1Mam (stock # 003551), B6129-TgN(MMTV-Cre)4Mam (stock # 003553), B6129-TgN(WAPCre)11738Mam (stock # 003552), B6;D2-TgN(Sycp1-Cre)4Min (stock # 003466), B6;FVB-TgN(GZMB-Cre)1Jcb (stock # 003734), B6;SJL-TgN(Co12a1-Cre)1Bhr (stock # 003554), BALB/c-TgN(CMV-Cre)#Cgn (stock # 003465), C.129P2-Cd19^{tm1(Cre)Cgn} (stock # 004126), C57BL/6-TgN(AlbCre)21Mgn (stock # 003574), C57BL/6-TgN(Ins2Cre)25Mgn (stock # 003573), C57BL/6-TgN(Zp3-Cre)3Mrt (stock # 003394), C57BL/6-TgN(Zp3-Cre)93Knw (stock # 003651), C57BL/6-TgN(Mx1-Cre)1Cgn (stock # 003556), DBA/2, TgN(xstpxLacZ)36And (stock # 002981), FVB/N-TgN(ACTB-Cre)2Mrt (stock # 003376), FVB/N-TgN(EIIa-Cre)C5379Lmgd (stock # 003314), FVB/N-TgN(Zp3-Cre)3Mrt (stock # 003377), STOCK Mttp^{tm1Sgy}Ldlr^{tm1Sgy}Apob^{tm1Sgy}Tg(Mx-Cre)1Cgn (stock # 004192), STOCK TgN(Wnt1-GAL4)11Rth (stock # 003829), STOCK TgN(Wnt1-Cre)11Rth (stock # 003829), STOCK TgN(balancer1)2Cgn (stock # 002858), STOCK TgN(balancer2)1Cgn (stock # 002859),and STOCK TgN(hCMV-Cre)140Sau (stock # 002471). Among these mice, B6.Cg(SJL)-TgN(NesCre)1Kln (stock # 003771), B6.Cg-Tg(Syn1-Cre)671Jxm (stock # 003966), and C57BL/6-TgN(Ins2Cre)25Mgn (stock # 003573) are examples of mice that have tissue specific Cre promoters. The B6.Cg-TgN(LckCre)548Jxm (stock # 003802) mice place Cre under control of the Lck promoter and do not have tissue specificity. The B6.FVB-TgN(EIIa-Cre)C5379Lmgd (stock # 003724) and BALB/c-TgN(CMV-Cre)#Cgn (stock # 003465) also have Cre recombinase under the control of a non-tissue-specific promoter. The disclosed floxed AR mice may be crossed with any of the Cre mice available to take advantage of additional promoter activity and specificity. Commercially available mice (Jackson Laboratory, Bar Harbor, Me.) that utilize the Flp recombinase expression system are 129S4/SvJaeSor-Gt(ROSA)26Sor^{tm1(FLP1)Dym} (stock # 003946) and B6;SJL-TgN(ACTFLPe)9205Dym (stock # 003800). Also disclosed are the Offspring of the disclosed floxed AR mice crossed with the disclosed Cre mice.

Disclosed are methods of evaluating treatment for cancer xenografts in ovarectomized mice comprising injecting cells wherein the cell has had the AR loci disrupted.

Disclosed are methods of evaluating treatment for cancer xenografts in ovarectomized mice comprising injecting cells wherein the cell has had the AR loci disrupted and wherein in the evaluation is of breast tumors, wherein the injected cells are, for example, MCF-7 cells, ZR-75-1 cells, or T47-D cells.

Disclosed are methods of evaluating treatment for cancer xenografts in ovarectomized mice comprising injecting cells wherein the cell has had the AR loci disrupted and wherein the evaluation is of ovarian tumors, wherein the injected cells are, for example, OVCAR-3 cells, ES-2 cells, or SKOV-3 cells.

Disclosed are methods wherein the ovarectomized mice are nude mice.

Disclosed is an ovarectomized nude mouse comprising a xenograft.

Disclosed is an ovarectomized nude mouse comprising a xenograft wherein the xenograft comprises cells injected into the mouse.

Disclosed is an ovarectomized nude mouse comprising a xenograft a) wherein the xenograft comprises cells injected into the mouse and b) wherein the cells injected have a disrupted AR loci.

Disclosed is an ovarectomized nude mouse comprising a xenograft a) wherein the xenograft comprises cells injected into the mouse, b) wherein the cells injected have a disrupted AR loci, and c) wherein the cells injected comprise a breast tissue cancer cell line.

Disclosed is an ovarectomized nude mouse comprising a xenograft a) wherein the xenograft comprises cells injected into the mouse and b) wherein the cells injected have a disrupted AR loci, c) wherein the cells injected comprise a breast tissue cancer cell line, and d) wherein in the cell line is MCF-7, ZR-75-1, or T47-D.

Disclosed is an ovarectomized nude mouse comprising a xenograft a) wherein the xenograft comprises cells injected into the mouse, b) wherein the cells injected have a disrupted AR loci, and c) wherein the cells injected comprise an ovarian cancer cell line.

Disclosed is an ovarectomized nude mouse comprising a xenograft a) wherein the xenograft comprises cells injected into the mouse and b) wherein the cells injected have a disrupted AR loci, c) wherein the cells injected comprise an ovarian cancer cell line, and d) wherein in the cell line is OVCAR-3, ES-2, or SKOV-3.

Disclosed are methods of evaluating osteoporosis in ARKO mice.

Disclosed are methods of evaluating tumor formation in ARKO mice.

Disclosed are vectors for making AR knockout animals, such as mice. Disclosed are vectors comprising a region 1 for homologous recombination with the 5′UTR of the androgen receptor gene, a region of Exon 1 of the androgen receptor gene, a region encoding a selectable marker, and a region 2 for homologous recombination with intron 1 of the androgen receptor gene.

Disclosed are vectors, wherein region 1 is at least 300 nucleotides long, wherein region 1 is at least 750 nucleotides long, wherein region 1 is at least 1000 nucleotides long, wherein region 1 is at least 1100 nucleotides long.

Also disclosed are vectors, wherein region 1 comprises the sequence has at least 70%, 80%, 90%, or 95% homology to the 5′UTR.

Disclosed are vectors, wherein the region of exon 1 comprises the ATG site in exon 1, wherein the region of exon 1 comprises at least 50 nucleotides of exon 1, or wherein the region of exon 1 comprises at least 100 nucleotides of exon 1.

Also disclosed are vectors, comprising selectable markers, for example, wherein the selectable marker is a Neo marker.

Also disclosed are vectors, wherein region 2 is at least 300 nucleotides long, wherein region 2 is at least 750 nucleotides long, wherein region 2 is at least 1000 nucleotides long, wherein region 2 is at least 1100 nucleotides long.

Disclosed are vectors comprising a region 1 for homologous recombination with a first intron of the androgen receptor gene, a region of an exon of androgen receptor contiguous with the first intron, a, a region encoding a selectable marker, and a region 2 for homologous recombination with a second intron of the androgen receptor gene.

Also disclosed are vectors, wherein the selectable marker is a negative selection marker or wherein the selectable marker is a positive selection marker.

Disclosed are vectors comprising a region 1 for homologous recombination with intron 1 of the androgen receptor gene, a region of exon 2 of the androgen receptor gene, a region encoding a selectable marker, and a region 2 for homologous recombination with intron 2 of the androgen receptor gene.

Also disclosed are vectors comprising a region 1 for homologous recombination with intron 1 of the androgen receptor gene, a region of exon 2 of the androgen receptor gene, a region encoding a selectable marker, and a region 2 for homologous recombination with intron 2 of the androgen receptor gene, wherein the selectable marker is flanked by a region 3 and a region 4, wherein region 3 and region 4 are substrates for a recombinase.

Disclosed are vectors comprising a region 1 for homologous recombination with intron 1 of the androgen receptor gene, a region of exon 2 of the androgen receptor gene, a region encoding a selectable marker, and a region 2 for homologous recombination with intron 2 of the androgen receptor gene, wherein the region of exon 2 is flanked by a region 3 and a region 4, wherein region 3 and region 4 are substrates for a recombinase.

Also disclosed are vectors comprising a region 1 for homologous recombination with intron 1 of the androgen receptor gene, a region of Exon 2 of the androgen receptor gene, a region encoding a selectable marker, and a region 2 for homologous recombination with intron 2 of the androgen receptor gene, wherein the selectable marker is flanked by a region 3 and a region 4, wherein the region of exon 2 is flanked by the region 4 and a region 5, and wherein regions 3, 4, and 5 are substrates for a recombinase.

Disclosed are vectors, wherein region 1 is at least 300 nucleotides long, wherein region 1 is at least 750 nucleotides long, or wherein region 1 is at least 1000 nucleotides long.

Disclosed are vectors, comprising a region of exon 2 of the AR gene.

Disclosed are vectors comprising a region 1 for homologous recombination with intron 1 of the androgen receptor gene, a region of Exon 2 of the androgen receptor gene, a region encoding a selectable marker, and a region 2 for homologous recombination with intron 2 of the androgen receptor gene, wherein the selectable marker is flanked by a region 3 and a region 4, wherein the region of exon 2 is flanked by a region 5 and a region 6, and wherein regions 3, 4, 5, and 6 are substrates for a recombinase.

Disclosed are cells comprising any of the vectors or nucleic acid molecules disclosed herein.

Disclosed are cells, wherein the cell is a cell which can be cultured, wherein the cell is an ES cell, and/or wherein the ES cell is a mouse ES cell.

Also disclosed are cells comprising a disrupted AR gene.

Disclosed are cells, wherein the disrupted AR gene comprises sites for recombination by a recombinase, wherein the sites are lox sites, wherein the recombinase is cre recombinase, and/or wherein the disrupted AR gene comprises a variant of the AR gene.

Disclosed are mammals comprising the vector and/or cells disclosed herein.

Disclosed are mammals, wherein the mammal is bovine, ovine, porcine, primate, mouse, rat, hamster, or rabbit.

Disclosed are mammals, wherein the disclosed vector has integrated into the mammals genome, comprising an integrated nucleic acid.

Also disclosed are mammals comprising an expressable recombinase, wherein the recombinase is specific for regions 3, 4, 5, and 6 of the vector, as well as mammals comprising a disrupted AR gene.

2. Compositions and Methods Related to BRCA1 and AR Interactions

Disclosed are methods to determine if BRCA1 influences AR activity comprising a) transfecting DU145 cells with AR and BRCA1 and b) a reporter gene to monitor results.

Disclosed are methods to determine if BRCA1 influences AR activity comprising a) transfecting DU145 cells with AR and BRCA1 and b) a CAT reporter to monitor results.

Disclosed are methods to determine if BRCA1 influences AR activity comprising a) transfecting DU145 cells with AR and BRCA1 and b) a LUC reporter to monitor results.

Disclosed are methods to determine the interaction of BRCA1 and coactivators of AR, for example, wherein the coactivator is ARA70 or AR55 or any other co-activators.

Disclosed are methods to determine the influence of the 17β-Estradiol on AR comprising inducing AR via administration of 17β-Estradiol in the presence of prostate specific antigen.

The disclosed methods can further comprise steps of, for example, measuring cell proliferation, measuring Bc1-2 in cells wherein the AR loci has been disrupted.

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular AR is disclosed and discussed and a number of modifications that can be made to a number of molecules including the AR are discussed, specifically contemplated is each and every combination and permutation of AR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Androgen Receptor

Androgen receptor belonged to a superfamily of steroid hormone receptors was first subcloned in 1988 (Chang, 1988). It contains a N-terminal transactivation domain, a central DNA binding domain (DBD) and a C-terminal ligand binding domain (LBD) (Umesono, 1995). By forming a homodimer and taking into account of the ligand and coregulators, the androgen receptors interact and regulate the transcription of numerous target genes (Ing, 1992; Schulman, 1995; Beatp, 1996; Yeh, 1996; Glass, 1997, Shibata, 1997). Androgen is the strongest ligand of the androgen receptor. However, it is not the only ligand. Estradiol has been found to activate androgen receptor transactivation through the interaction with androgen receptor (Yeh, 1998). Besides, androgen and androgen receptor do not only act in male. The increasing evidence has displayed that the androgen and androgen receptor (AR) may also play important role in female physiological processes, including the process of folliculogenesis, the bone metabolism and the maintenance of brain functions (Miller, 2001).

Androgen is the most conspicuous amount of steroid hormone in ovary (Risch H A, 1998). The concentrations of testosterone and estradiol in the late-follicular phase when estrogens are at their peak are 0.06-0.10 mg/day and 0.04-0.08 mg.day respectively (Risch H A, 1998). The ratio of androgens versus estrogens in the ovarian veins of postmenopausal women is 15 to 1 (Risch, 1998; Doldi N, 1998). Androgen receptor is expressed dominantly in granulosa cells of ovary (Hiller S G, 1992; Hild-Petito S, 1991). With the overproduction of ovarian androgen, women with polycystic ovarian syndrome suffered from impairment of ovulatory function which is characterized with the increasing number of small antral follicles, but arrest in grafian follicles development (Kase, 1963; Futterweit W, 1986; Pache T D, 1991; Spinder T, 1989; Spinder T, 1989; Hughesdon P E, 1982). This symptom has suggested that AR may play a proliferative role in early folliculogenesis but turn to inhibitory effect in late folliculogenesis. The recent studies conducted in animals have supported this hypothesis (Harlow C R, 1988; Hilllier S, 1988; Weil S, 1998; Vendola K, 1998; Weil S, 1999; Vendola K, 1999). Administration of hihydroxytestosterone (DHT) in rhesus monkeys has increased the number of primary, preantral and small antral follicles. Since DHT is the metabolite of testosterone and cannot be aromatized, the result suggested the proliferative effect was through AR system (Vendola K, 1999).

2. Androgens and Bone

In the cartilage and bone system, androgen receptor (AR) had been shown to be expressed in chondrocytes, osteoblasts and osteocytes (Benz D J, 1991). Clinically, a number of studies suggested that combined therapy of estrogen plus androgen enhances bone mineral density and bone mass to a more significant degree than estrogen therapy alone in postmenopausal women (Watt N B, 1995; Castelo-Branco C, 2000; Davis S R, 1995). The mechanism of androgen act on bone system is controversial. Some studies suggest that the effect is mainly through the aromatase to transform the androgen to estrogen (Schweikert H U, 1980) However, in the other studies that administration of antiandrogens, including flutamide and Casodex, to female mice resulted in osteopenia and could not be reversed by aromatase inhibitors suggest the direct role of AR in bone metabolism (Goodram D, 1993; Lea C E, 1998). Recent evidence have suggested the AR and ER interaction, although the consequence of this interaction is unclear (Migliaccio A, 2000; Panet-Raymond V, 2000).

3. Androgens and Breast Cancer

Because the benefit of androgen therapy is not limited to premenopausal breast cancer patients, it appears that the effect of androgens on mammary tumor growth is not limited to an inhibitory effect on gonadotropin secretion. This conclusion is supported by the above mentioned breast cancer cell line studies which indicate a growth inhibitory role for androgen bound AR. One possibility is that androgen-AR modifies the estrogen responsiveness of breast cancer cells. DHT suppresses the level of ER mRNA and protein in human ZR-75-1 breast cancer cells (29). DHT treatment also increases 17β-hydroxysteroid dehydrogenase activity in this cell line, resulting in an increased conversion of estradiol to estrone, which has lower estrogenic activity (30). Alternatively, it has recently been demonstrated that DHT down regulates the expression of bc1-2 in breast cancer cells (31). Bc1-2 acts by inhibiting cell death (32), therefore, androgens may sensitize breast cancer cells to apoptosis. Androgens, acting through AR, have also been demonstrated to enhance the expression of the cyclin-dependent kinase (CDK) inhibitor p21(WAF1/CIP1) (33). Unlike bc1-2, which functions in the regulation of apoptosis, the expression of p21(WAF1/CIP1) is implicated in cell cycle arrest (34) and in the withdrawal of cells from the cell cycle during differentiation (35). The lack of p21(WAF1/CIP1) alleles in a human cancer cell line completely abrogated G1 arrest in response to DNA damage (36). These observations suggest that the reduction of proliferation of breast cancer cells by androgens may be partly mediated through sensitivity to apoptosis and cell cycle control.

a) AR and the Tumor Suppressor Gene BRCA1.

While most cases of breast cancer are diagnosed in women without a family history of the disease, it has long been recognized that a family history is a major risk factor for breast cancer, representing 5-10% of all cases. Mutations of the BRCA1 gene account for approximately 45% of familial breast cancer and up to 80% of families with both breast and ovarian cancer (37,38). The function of BRCA1 is not yet fully understood. The 1863 amino acid BRCA1 protein does not resemble any other protein of known function but has been implicated in genome stability, DNA repair, cell cycle control, and transcriptional activation. The tumors of BRCA1 mutation carriers are characterized by a high degree of chromosomal aberrations, as well as the somatic loss of the wild type chromosome 17q (where the BRCA1 gene is located), compared to sporadic mammary tumors (39). It is unclear if loss of heterozygosity is a prerequisite for further chromosomal alterations. BRCA1 interacts with Rad51, a protein required for mitotic stability and the repair of double stranded DNA breaks (40). The proposed involvement of BRCA1 in cell cycle control derive from the observation that ectopic expression of BRCA1 induces the expression of the cyclin dependent kinase inhibitor p21(Waf1/Cip1), leading to cell cycle arrest (41). The inhibition of BRCA1expression using antisense nucleotides results in an acceleration of mammary epithelial cell growth. BRCA1 expression is induced during the late G1/early S phase of the cell cycle and is phosphorylated in a cell cycle dependent manner (42,43), also suggesting an involvement in cell cycle progression.

4. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

5. Hybridization/selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, New York, 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

6. Nucleic Acids

There are a variety of molecules disclosed herein that are nucleic acid based, including for example the nucleic acids that encode, for example AR, or any of the nucleic acids disclosed herein for making AR knockouts, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed MRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxynbose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example AR, or any of the nucleic acids disclosed herein for making AR knockouts, all of which are encoded by nucleic acids or are nucleic acids. The sequences for the human analogs of these genes, as well as other anlogs, and alleles of these genes, and splice variants and other types of variants, are available in a variety of protein and gene databases, including Genbank (for example Genbank accession numbers NM_{—}000044). Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as the AR gene as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the AR gene typically will be used to produce an amplified DNA product that contains the a region of the AR gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as AR, and the nucleic acids used for the generation of AR knockouts, or the genomic DNA of any of the disclosed nucleic acids, such as AR, and the nucleic acids used for the generation of AR knockouts or they can interact with the polypeptide encoded by any of the disclosed nucleic acids, such as AR, and the nucleic acids used for the generation of AR knockouts. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

7. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991)Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed AR nucleic acids or vectors for example, lipids such as liposomes, such as cationic liposomes (e.g., DOTMA, DOPE, DC-holesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

In the methods described above which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used gene transfer methods.

As one example, if a nucleic acid disclosed herein is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

a) In Vivo/ex Vivo

As described above, the compositions can be administered in a pharmaceutically acceptable carrier and can be delivered to the subject s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

8. Expression Systems

The nucleic acids that are delivered to cells typically contain expression controlling systems. For example, the inserted genes in viral and retroviral systems can contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Baneiji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR- cells and mouse LTK- cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

9. Peptides

a) Protein Variants

As discussed herein there are numerous variants of the AR protein that are known and herein contemplated. In addition, to the known functional AR allelic variants there are derivatives of the AR proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | AlaA |
| Allosoleucine | AIle |
| Arginine | ArgR |
| Asparagines | AsnN |
| aspartic acid | AspD |
| Cysteine | CysC |
| glutamic acid | GluE |
| Glutamine | GlnQ |
| Glycine | GlyG |
| Histidine | HisH |
| Isolelucine | IleI |
| Leucine | LeuL |
| Lysine | LysK |
| Phenylalanine | PheF |
| Proline | ProP |
| pyroglutamic acidp | Glu |
| Serine | SerS |
| Threonine | ThrT |
| Tyrosine | TyrY |
| Tryptophan | TrpW |
| Valine | ValV |

TABLE 2
Amino Acid
Substitutions

| | Exemplary Conservative |
|---|---|
| | Substitutions, others are |
| Original Residue. | known in the art |
| Ala | Ser |
| Ar | glys, gln |
| Asn | gln; his |
| Asp | Glu |
| Cys | Ser |
| Gln | asn, gln |
| Glu | Asp |
| Gly | Ala |
| His | asn; lys |
| Ile | leu; val |
| Leu | ile; val |
| Lys | arg; gln; |
| Met | Leu; ile |
| Phe | met; leu; tyr |
| Ser | Thr |
| Thr | Ser |
| Trp | Tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr, Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of AR and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, 3. MoL Biol. 48: 443 (1970), by the search Is for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

10. Antibodies

Disclosed are antibodies related to the disclosed compositions. For example, it is understood that the disclosed knockouty mice could be used for generation of a particular antibody, could produce antigens which would be desirable in the generation of antibodies, such as a monoclonal antibody, and could have antibodies administered to them. Those of skill in the art understand how to generate monoclonal antibodies and administer them, for example, see Kohler and Milstein, Nature, 256:495 (1975) which is herein incorporated by reference for material related to antibody production.

11. Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffier, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyrivic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

12. Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

13. Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

14. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for assessing testing compounds related to androgen receptor comprising the ARKO mouse disclosed herein, and the reagents to aid in the testing.

D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert -butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J.Biol.Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence of an AR exon, such as exon 2, and sequence recognized by a recombinase enzyme.

Also disclosed are nucleic acid molecules produced by the process comprising linking in an operative way a nucleic acid molecule comprising a sequence having 80% identity to a sequence of an AR exon, such as exon 2, and sequence recognized by a recombinase enzyme.

Disclosed are nucleic acid molecules produced by the process comprising linking in an operative way a nucleic acid molecule comprising a sequence that hybridizes under stringent hybridization conditions to a sequence of an AR exon, such as exon 2, and sequence recognized by a recombinase enzyme.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate, such as a human, monkey, ape, chimpanzee, or orangutan.

Also disclose are animals produced by the process of adding to the animal any of the cells disclosed herein.

Disclosed compositions and methods, such as vectors, that can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

Disclosed are nucleic acids used to modify a gene of interest that is cloned into a vector designed for example, for homologous recombination.

E. Methods of Using the Compositions

1. Methods of Using the Compositions as Research Tools

The disclosed compositions can be used in a variety of ways as research tools. For example, the disclosed compositions, such as the ARKO mice can be used to study reagents related to prostate cancer, as well as being used to generate tissue specific knockouts of AR, such as a mammary or prostate or liver specific knockout. The disclosed compositions can also be used as diagnostic tools related to diseases such as prostate cancer, and any disease related to androgen receptor function.

Disclosed are methods of producing a tissue specific androgen receptor knockout comprising mating a disclosed androgen receptor knockout mouse with a mouse that contains a tissue specific promoter controlled recombinase.

Disclosed are methods of testing the effect of a composition on a cell or an animal comprising incubating the composition with one or more of the disclosed androgen receptor knockout cell lines or androgen receptor knockout animals.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Generation of Mice homozygous for Floxed AR Exons

a) General Procedural Protocol for Generation of Mice for General and Tissue Specific Targeted Disruption of AR.

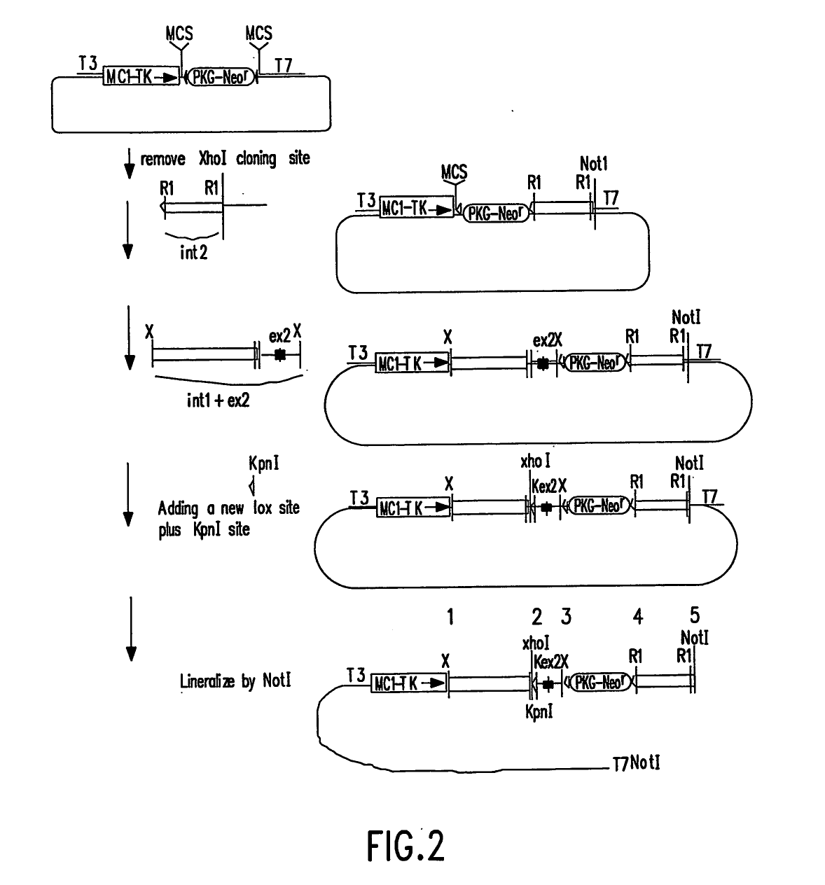

To generate a general and mammary specific homologous recombinants of the AR gene, which could be used in general and specific knockout analysis, a loxP-Cre strategy was used. The loxP-Cre system utilizes the expression of the P1 phage Cre recombinase to catalyze the excision of DNA located between flanking lox sites (53). This strategy differs from the standard targeted disruption procedure in that ES cells are generated in which the targeted segment is not disrupted but flanked by lox sites (floxed). The targeted gene thus functions normally and mice can be bred to homozygosity for the targeted locus. The floxed locus is disrupted by crossing the floxed strain to a strain transgenic for a Cre recombinase transgene under the control of a tissue specific or general promoter. The floxed locus in the progeny will function normally in all tissues except those that express Cre recombinase causing recombination between the loxP sites and disruption of the floxed locus. To disrupt the AR gene, exon 2 has been targeted for lox/Cre mediated excision FIG. 2). Exon 2 encodes the first zinc finger of the AR DNA binding domain. The deletion of exon 2 is expected to cause a frameshift resulting in a truncated protein containing the AR N-terminal activation domain (encoded by exon 1) and a stretch of 14 missense amino acids before a stop codon. FIG. 2 depicts the construction of the AR targeting vector. Genomic clones of the mouse androgen receptor (mAR) were isolated from a bacteriophage lambda genomic library constructed from 129 ES cells (University of Rochester Transgenic Core Facility) using the mAR exon 2 sequence as a probe. Exon 2 and flanking regions were cloned into the PKI vector as shown in FIG. 2. A thymidine kinase selectable marker (MCT-TK) was inserted at the 5′ end of the multiple cloning site. A neomycin resistance cassette (PGK-Neo^{r}) flanked by two lox sites was inserted into the middle of the multiple cloning site dividing the multiple cloning site into two parts. A 3 kb fragment of intron 2 was introduced into the 3′ EcoRI of the multiple cloning site (FIG. 2). A 5 kb fragment containing intron 1 and exon 2 was cloned into the 5′ XbaI site. Finally, a lox site was cloned at the 5′ end of exon 2 (FIG. 2). The plasmid insert was verified by DNA sequencing. The targeting vector can be linearized using a unique NotI site.

The floxed AR carrying mice were crossed to transgenic mice expressing Cre from the β-actin promoter (ACTB-Cre, commercially available from the Jackson Laboratory). The ACTB-Cre transgene is expressed in all cells by the blastocyst stage of embryogenesis. Prior to the ovarian and bone analysis, analysis of the AR exon 2 targeted deletion by Southern blot and PCR analysis was conducted to determine the extent of recombination. For bone analysis the extent of recombined AR, osteoblasts can be isolated from neonatal calvaria and osteoclasts from neonatal long bones.

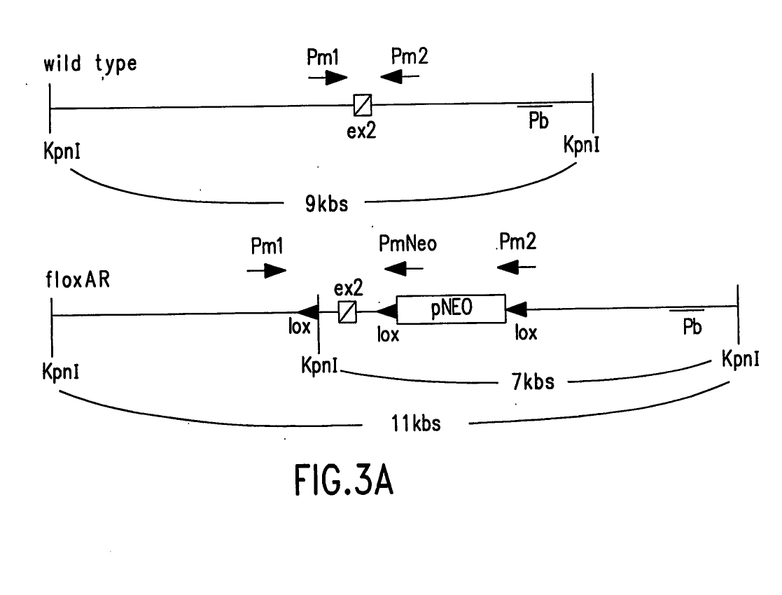

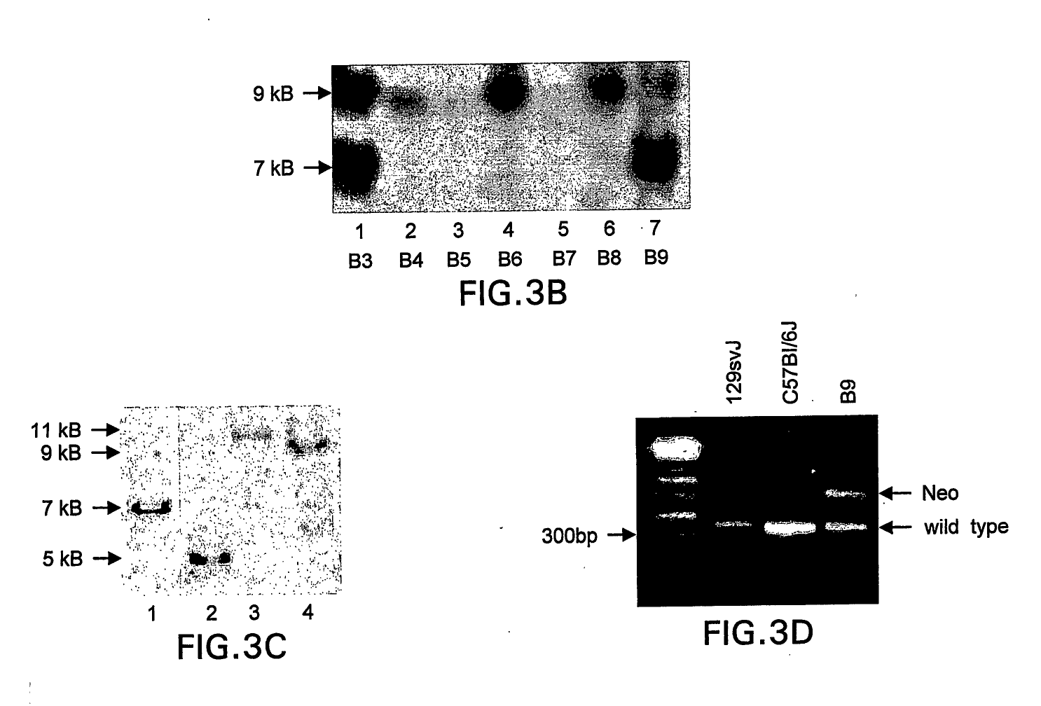

The AR targeting vector was linearized and electroporated into male ES cells derived from 129SVJ mice essentially as previously described (53). Neomycin resistant colonies were selected in 300 μg/ml G418. Identification of homologous recombinant ES cells was performed as shown in FIG. 3 by Southern blot and PCR ES cell clones containing the homologous insertion of the targeting vector into the AR locus were electroporated with a pCMV-Cre expression plasmid to remove the neomycin selection cassette. FIG. 3D shows DNA isolated from recombinated flox AR-ES cells, from 129svJ and from C57B1/6J ES cells are amplified using the Pm1 and PmNeo PCR primers. The flox AR locus yields a 600 bp fragment and the uninterrupted AR locus yields a 400 bp band. FIG. 3C shows the Southern blot analysis to identify the ES cell clones in which only the neomycin cassette, but not the desired AR sequences, has been removed. The desired ES clones containing the AR exon 2 flanked by loxP sites (floxed) was microinjected into 3.5 day C57B1/6J blastocysts. The resulting chimeras were identified by coat color chimerism.

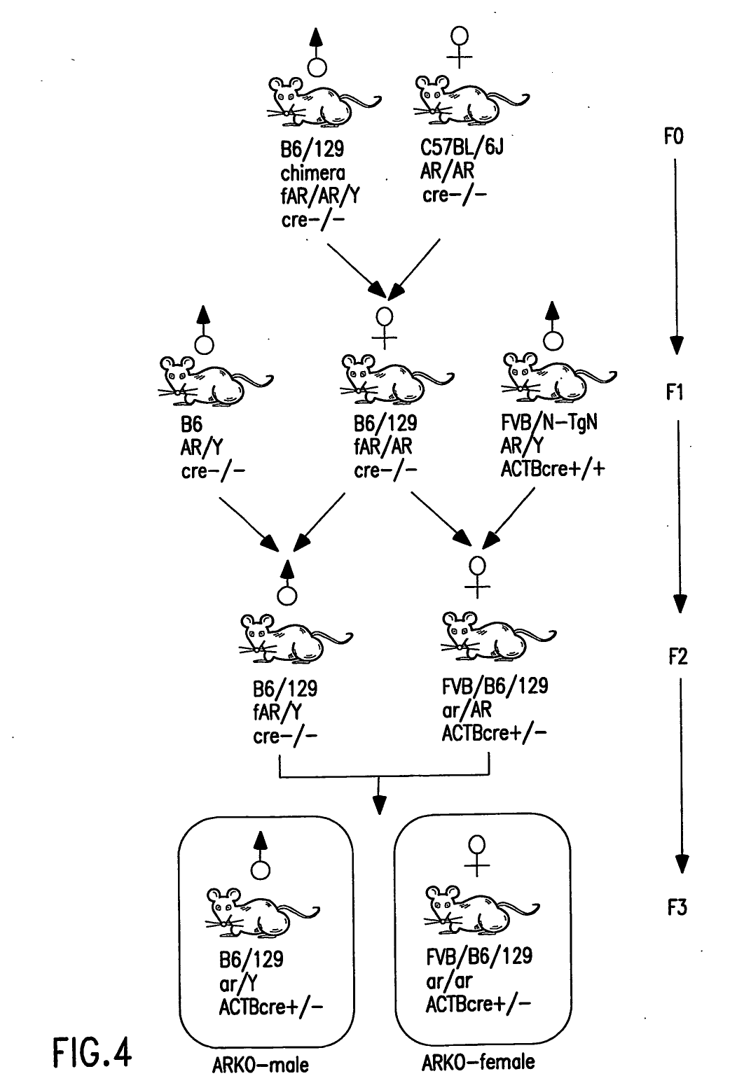

The mating strategy was illustrated in FIG. 4. The strain of the mosaic founder was C57BL/6-129/SVJ. The mating between the founder and the female B6 mice create agouti female offspring carrying the heterozygous floxAR (F1). The F1 offspring would mate with the B6 male mice to create male mice carrying the floxAR in X chromosome (F2). They would also mate with the homozygous ALTB cre male mice that carrying the cre-recombinase under the control of mactin promoter to generate female mice carrying both the heterozygous floxAR and cre recombinase (F2). Mating these two genotypes of the F2 mice together finally generated female mice carrying the homozygous floxAR and cre recombinase. The β-actin promoter driven cre recombinase would work to delete the floxAR fragment in all the cells.

b) Experimental Procedures

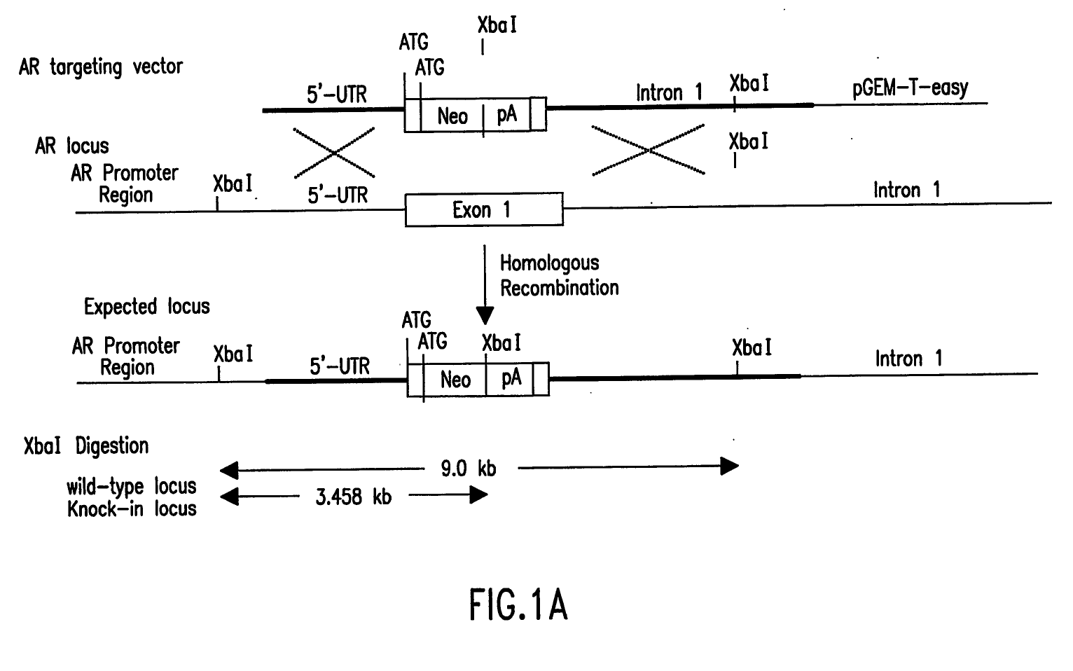

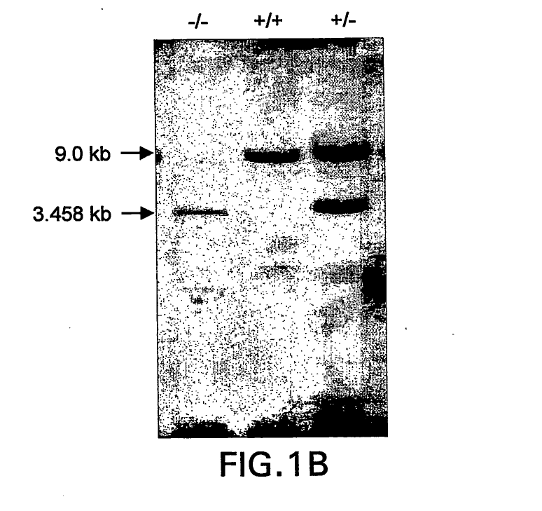

The steps leading to the birth of the female mice carrying the cre recombinase and the homozygous floxAR genes in both X chromosomes are illustrated in FIG. 1-4. We first constructed the targeting vector followed by generation of the founder mice carrying the floxAR fragment. The founder mice were then mated with the Female cre-recombinase mice to generate the F3 offspring that carries the homologous floxAR and cre recombinase.

(1) Construction of Targeting Vectors

Two genomic clones containing exon 2 of mouse AR (mAR) were isolated from an ES129 bacteriolphage λ genomic library (Strategene) by using the mAR exon 2 sequence as the probe see the underlined nucleotides of SEQ ID NO:10, setting forth the exon 2 sequences with the flanking intron sequence. The flanking region was sequenced and cloned into the PKI vector. FIGS. 1 and 2 detail the procedure for the construction of targeting vectors.

(2) Generation of the Chimera Founder Mice

The ES cell line 129/SVEV derived was grown according to the conditions described previously (33). For electroporation, 40 μg of the targeting vector was linearized by NotI and suspended together with 10⁹ ES cells in 1 ml of Dulbecco's modified Eagle's medium. The cells were electropolarized at 300 F, 0.4 msec. (GenePulsar II System, BioRad,). The neo^{r} colonies were selected in the presence of 300 μg of G418 per ml. Homologous recombinations were identified by genomic Southern blot hybridization. The clones with homologous recombination were amplified and re-electropolarized to introduce pCMV cre-recombinase vector into the cells. The transient expression of the cre recombinase in the cells resulted in three types of recombination, which could also be checked by Southern blot hybridization. (Kaczmarczyk S J, Green J E. A single vector containing modified cre recombinase and LOX recombination sequences for inducible tissue-specific amplification of gene expression. Nucleic Acids Res. 2001 Jun 15;29(12):E56-6.). The ES cells with type I recombination would then be injected into the inner cell mass of blastocysts which would be implanted to the uterus of foster mothers for further development and birth.

(3) Mating of the Chimera Founder Mice With the Homozygous Cre Mice

The mating strategy is illustrated in FIG. 4. The strain of the mosaic founder was C57BL/6-129/SVJ. The mating between the founder and the female B6 mice created agouti female offspring carrying the heterozygous floxAR (F1). The F1 offspring mate with the B6 male mice to create male mice carrying the floxAR in the X chromosome (F2). They were also mated with the homozygous ALTB cre male mice that acarry the cre-recombinase under the control of a β-actin promoter to generate female mice carrying both the heterozygous floxAR and cre recombinase (F2). Mating these two genotypes of the F2 mice generated female mice carrying the homozygous floxAR and cre recombinase. β-Actin is a house keeping gene and it universally expresses in every tissue, Therefore, the β-actin promoter driven cre recombinase works to delete the floxAR fragment in all the cells.

(4) Primer Design and Genotyping of AR KO Mice.

Based on the sequence information obtained for the AR genomic DNA (see SEQ ID NO:10), two pairs of primers have been designed to distinguish the wild type AR, ARKO, and floxed AR X chromosome on mice.

For examining the floxed AR X chromosome: the 5′ primer is named “select” which is located in the intron 1 and its sequence is 5′-GTTGATACCTTAACCTCTGC-3′, the 3′ end primer is 2-9 which is located in intron 2 and its sequence is: 5′-CCTACATGTACTGTGAGAGG-3′. If the mice carry floxed X, the PCR product size from this pair of primers would be 238 bp. If the mouse is wild type, this pair of primers will amplify a PCR product with 580 bp. For examining the floxed AR X chromosome, primer “select” and primer“2-3” are used. 2-3 primer is the 3′-end primer which is located in the exon 2 with sequence: 5′-TTCAGCGGCTCTTTTGAAG-3′. This pair of primers will amply a product with 444 bp. The expression of Cre and the internal control IL2 were confirmed by PCR during genotyping. The primer design and PCR conditions followed Jackson lab suggestion.

c) Results

(1) Construction of Targeting Vectors and Electroporation of the ARKO Plasmid in ES Cells

To disrupt the AR gene, exon 2 was targeted for loxP/Cre mediated excision. Exon 2 encodes the second zinc finger of the DBD and deletion of the DBD has been reported to result in the complete androgen insensitivity. As shown in FIGS. 1 and 2, the PKI vector was modified from the pBluescript vector and a thymidine kinase selective marker (MCT-TK) was inserted at the 5′ end of the multiple cloning site. The other neomycin resistant marker (PKG-Neo^{r}) flanked by two lox sequences was inserted at the middle of the multiple cloning site that separates the multiple cloning site into two parts. The XboI cloning site was filled in using klenow fragment. Two fragments, one was 3 kb fragment with the intron 2 sequence and the other was a 5 kb fragment including the 3′ end of intron 1, exon 2 and 5′ end of intron 2 sequence, were generated using the extended high fidelity polymerase chain reaction system. (see SEQ ED NO:10) The loxP-Cre system utilizes the expression of the P1 phage Cre recombinase to catalyze the excision of DNA located between flanking lox sites. This strategy differs from the standard targeted disruption procedure since the ES cells were generated in which the targeted segment is not disrupted but flanked by lox sites (floxed). The arrangement of loxP sites located in intron 1 and intron 2 can preserve the AR function before introduction of Cre. After selection and screening for homologous recombinants, a pCMV-Cre expression plasmid was then transiently transfected into the ES cell clones to induce recombination between any two loxP sites.

(2) Screening of ARKO ES Cells

For the screening of the ES cell clone with ARKO, two pairs of primers were designed to distinguish between the wild type and floxed AR locus (FIG. 2A). Southern blotting was applied to verify the floxed AR construct in ES cells (FIG. 2B). The transient transfection of pCMV-Cre resulted in type 1, type 2 and type 3 recombinations. As illustrated in FIG. 2C, ARKO recombinates were obtained. This type 2 recombinant (determined by Southern blot analysis) containing loxP sites flanking the AR exon 2 was then used for the blastocyst injection to generate floxed AR-chimera male mice.

(3) Genotype Screening of Floxed AR-chimera Male Mice

Two pair of primers were applied for genotype screening. As shown in FIG. 2D, the floxed AR-chimera mice show longer PCR products. With 2 separate blastocyst injections, we were able to obtain 4 individual floxed AR-chimera male mice.

(4) Generation of ARKO Mice

To generate mice with the disruption of AR, the floxed AR male mice were crossed to female mice carrying Cre under the control of the beta-actin promoter (ACTB). ACTB-Cre transcription will be activated in all tissues to generate mice lacking functional AR. As shown in FIG. 4, after two matings, F2 mice were obtained with male ARKO mice (floxed AR/Y male) and heterozygous female ARKO on one X chromosome (ar/AR-ACTB/Cre female). These mice were then bred together to obtain F3 ARKO female (ar/ar-ACTB/Cre female) and male (ar/Y-ACTB/Cre male) mice (FIG. 4).

(5) Genotype Screening of ARKO Mice.

Mating of floxed AR female and ACTBcre male mice generates pups of four possible genotypes (ar/Y-ACTB/Cre-male, ar/AR-ACTB/Cre-female, AR/Y-ACTB/Cre-male, AR/AR ACTB/Cre-female) with ratio of 1:1:1:1.

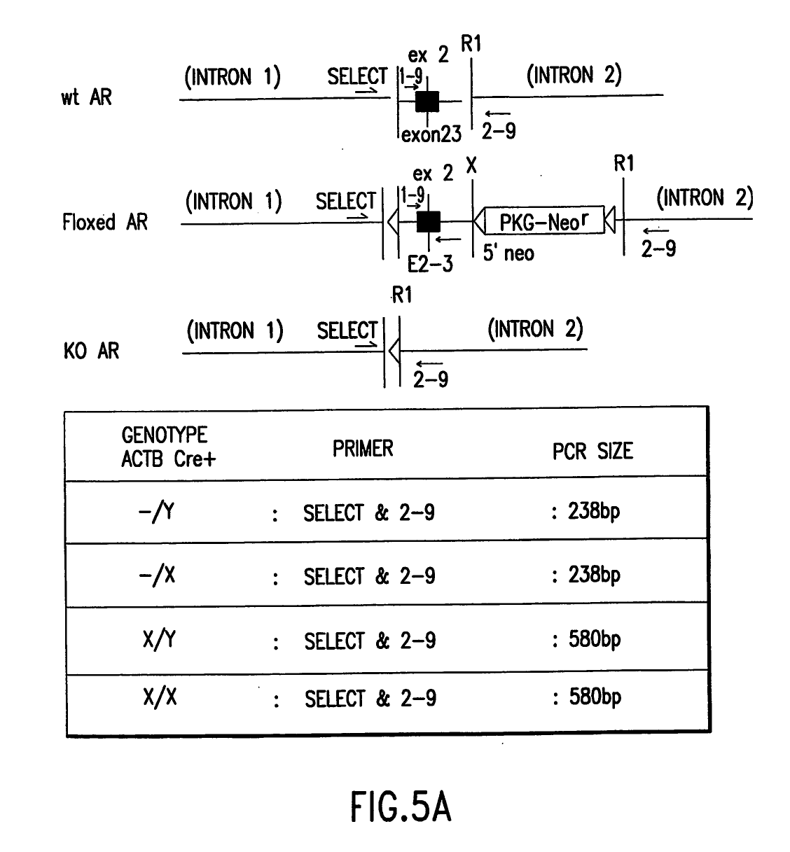

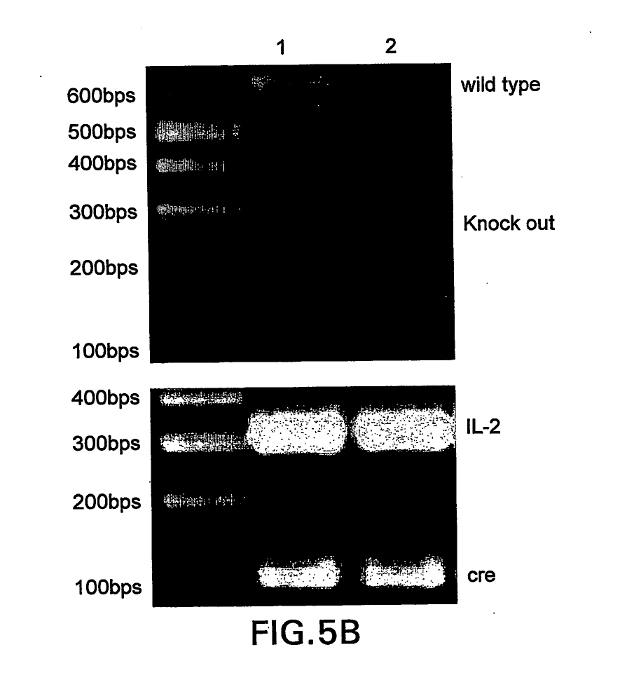

Three primers, select, exon 2-3, and 2-9 (for the relative position of each primer in the AR gene see FIG. 5A), were synthesized to amplify mice genomic DNA to distinguish the Flox AR mice, ARKO mice and wild-type mice. As shown in FIG. 5, ARKO male mice using select and 2-9 primers to PCR amplify the 238 bp DNA were identified. In contrast, wild-type mice produced 580 bp PCR amplified DNA fragment using select and 2-9 primers (FIG. 5B).

(6) Phenotype Characterization of ARKO Male Mice

Six 5-week-old ARKO mice were sacrificed for the comparison of their phenotype with wild-type male and female mice. ARKO male mice had female-like appearance and body weight. The genitoanal distance of 0.55 cm is similar to female mice, yet is shorter than the wild-type mice at 1.05 cm. The ARKO male mice have gonads that look like testis, yet the size is much smaller, only 20% as compared to the same age of wild-type male mice. The results were compared among siblings.

For 8-week-old mice, the results indicate that the male KO mice have female like outlook and body weight. The genitoanal distance of AR KO male is 0.59 cm, which is shorter than wild type male sibling (1.12 CM) and similar to their female siblings. Similar to 5-week-old mice, we also observed that male ARKO mice have gonads and the outlook is like testis, but the size only 20% of that of wild type male sibling.

(7) Male Reproductive Organs

The prostate, seminal vesicles, epididymides and vas deferens were absent in the ARKO male mice which is similar in Tfm mice or humans with complete androgen insensitivity.

(a) Testes

Testis in Tfm mice are smaller (20% of normal), cryptorchid, and composed of immature tubular elements surrounded by several layers of peritubular cells and enlarged Leydig cells. The number of Leydig cells are normal or slightly reduced. The reduced seminiferous tubules contain only Sertoli cells, spermatogonia and primary spermatocytes. Spermatogenesis arrested at the spermatocyte stages or earlier. In older Tfm mice, the Leydig cells apeared to be hypertrophied.

(b) Bone

Five-week-old ARKO male mice have no ovious change in the bone structure. It may be due to the mice are pre-puberty. However, in 8-week-old ARKO male mice with the DEXA study revealed that the bone density in the ARKO mice is decreased. After fixatation, decalcification and staining, 8-week-old ARKO mice were observed to have cell number changes with increases in osteoblasts and even higher number increases of osteoclasts, which is consistent with reduced bone density in ARKO male mice. This result is compatible with the results of previous studies that further strengthen the roles of AR in bone metabolism. Lea et al. has reported that the antiandrogen Casodex inhibited the protective effects of androstenedione on ovariectomy-induced bone loss, whereas administration of an aromatase inhibitor was ineffective. Furthermore, the skeletal effects of castration in the male animals can be prevented by either administration of T or the nonaromatizable androgens (Lea, O. A, Kvinnsland, S., and Thorsen, T. (1989) Cancer Research 49, 7162-7167). Furthermore, the skeletal effects of castration in the male animals can be prevented by either administration of testosterone or the nonaromatizable androgens (Kapitola J, 1995; Somjen D, 1994; Turner R T, 1990; Wakley G K, 1991). The results disclosed herein prove that androgen and androgen receptor play a role in bone metabolism.

(c) Adipose Tissue and Obesity

Five-week-old ARKO male mice have no ovious change in the adipose tissue. However, 8-week-old ARKO male mice start to show the increased size of white adipocytes in subcutaneous and intrarenal regions, suggesting that AR can play a role in the sexual dimorphism of fat distribution. Women with abdominal fat distribution can have increased percentage of free T in the peripheral blood. In contrast, obesity in men may be characterized by reduced T.

(8) ARKO Female Mice

Due to infertility of Tfm male mice, it is difficult to generate ARKO female mice to study the roles of AR in female tissues. The floxed AR male mice, can now be mated with ACTB-Cre ar/AR-female to generate ARKO in female mice. 12 ARKO female mice were obtained. Potential defects in fertility can be detected as part of the analysis. Male Tfm mice that lack a functional AR due to mutation suffer from testicular feminization and complete androgen insensitivity. However, the other organ systems of the Tfm mice are apparently normal. Female ARKO mice can demonstrate a fertility defect and have an increased susceptibility to osteoporosis, but be otherwise developmentally normal. Necropsies can be performed on adult ARKO female mice and wild type female littermates to examine all organ systems in terms of gross morphology and histology.

(a) Breeding Experiments.

A short term breeding analysis was performed to determine that the ARKO female mate. Five ARKO and five wild type females at six weeks of age were individually housed with known fertile wild type males. Animals were paired for two weeks and females will be examined for the presence of copulatory plugs daily. Mating ARKO females were involved in continuous breeding studies. In those studies, ten ARKO females and ten wild type females were individually paired with known fertile wild type males. Females were considered infertile if they have not given birth after two months of continuous pairing. Fertile pairs are housed together for six months. The number of litters born and the number of pups per litter were compared between the wild type and ARKO females.

(b) Female Ovary

AR is expressed predominantly in the granulosa cells of the ovary, although elevated AR protein and mRNA is also observed in early and midluteal phase luteal cells (69,70). Although testosterone is the biosynthetic precursor of estrogen, there is evidence that androgens, acting through AR, may participate in ovarian function. In rhesus monkeys, administration of testosterone, dihydroxytestosterone (DHT), increases the number of primary follicles and enhances the level of IGF-1 and the IGF-1 receptor in primary follicle oocytes (73). DHT is the metabolite of testosterone and cannot be aromatized to estrogen. The ability of DHT to stimulate the initiation of follicular development suggests that this phenomena is mediated by AR rather than metabolism of testosterone (73). Testosterone has been shown increase the mRNA of the FSH receptor in granulosa cells in primary, preantral, periantral, and antral follicles, suggesting that androgens may serve to amplify the effect of FSH (71). Androgens are elevated in patients suffering from polycystic ovarian syndrome (PCOS). PCOS is characterized by suppression of follicular maturation, although it remains unclear whether the hyperandrogenism observed in these patients is the cause or effect of follicular arrest (reviewed in (74)). These observations suggest that AR is functionally important in the ovary.

The AR floxed mice were crossed to mice expressing Cre recombinase under the control of the human β-actin promoter (ACTB-Cre, commercially available from the Jackson Laboratory) as shown in FIG. 4. This transgene was characterized to be expressed in all cells of the embryonic blastocyst. ACTB-Cre, floxed AR females therefore lacked a functional AR in all tissues, including the ovary. Using this strategy, genetically male mice carrying the floxed AR and the ACTB-Cre transgene lacked a functional AR and therefore appeared externally female, as is the case for male Tfm mice that carry a nonfunctional mutated AR gene. To distinguish genetically female and male mice, tail biopsy DNA was screened by PCR for the Y-linked gene Sry (75). Female mice of the same genetic background carrying two non-disrupted AR alleles are used as controls for all experiments. The reproductive phenotype of the ARKO female mice was assessed through continuous breeding experiments to known fertile wild type males. The number of pups per litter and the number of litters was scored and compared to those of wild type females. Although the female mice in the study were fertile, the average number of the pulps per litter is significantly less than the wild type. The significance is even obvious as the age of the female mice increase and as in the mice receiving superovulation for induction.

Although the female mice in our study were fertile, the average number of the pups per litter is significantly less than the wild type. This significance is even more obvious as the age of the female mice increases and in the mice receiving superovulation for induction of ovulation. The histological examination in the mice revealed that the induction number of oocytes deceased and the oocytes depleted faster in the ARKO mice than in the wild type mice. Previous studies have revealed that androgen played a positive role in the early stage of folliculogenesis (Weil S, Vendola K, Zhou J, Bondy C A. Androgen and follicle-stimulating hormone interactions in primate ovarian follicle development. J Clin Endocrinol Metab. 1999 Aug;84(8):2951-6; Vendola K, Zhou J, Wang J, Famuyiwa O A, Bievre M, Bondy C A. Androgens promote oocyte insulin-like growth factor I expression and initiation of follicle development in the primate ovary. Biol Reprod. 1999 Aug;61(2):353-7.). However, it is unclear if the androgen effect is mediated via AR. For example, Vedola et al. suggested that this effect could be mediated via growth factor signal pathways instead of the AR-mediated pathway (Vendola K, Zhou J, Wang J, Famuyiwa O A, Bievre M, Bondy C A. Androgens promote oocyte insulin-like growth factor I expression and initiation of follicle development in the primate ovary. Biol Reprod. 1999 Aug;61(2):353-7). However, the decreasing number in primary, preantral and small antral follicles in the ARKO mice suggested that AR may also play a role in this process. It is possible that AR's roles could be compensated for because there were still many follicles spared which can then mature.

(c) Female Breast

The morphology and histology of breast in our ARKO mice showed no major difference as compared to wild type mice. Early studies suggested that androgen-AR may play some roles in the breast cancer progress (Bentel J M, Birrell S N, Pickering M A, Holds D J, Horsfall D J, Tilley W D. Androgen receptor agonist activity of the synthetic progestin, medroxyprogesterone acetate, in human breast cancer cells. Mol Cell Endocrinol. 1999 Aug 20;154(1-2):11-20.). The detailed mechanisms, however, remain unclear. The ARKO female mice can provide an in vivo model to study how carcinogens induce breast tumor in the presence versus absence of AR in breast tissues. The mammary specific androgen receptor knock-outs discussed herein can also confirm these results.

(d) To Investigate the Influence of AR in a Mouse Model of Osteoporosis Using Female Mice Lacking a Functional AR.

The female ARKO mice can be used to examine the role of AR in osteoporosis. Clinically, a number of studies suggest that combined therapy of estrogen plus androgen enhances bone mineral density and bone mass to a more significant degree than estrogen therapy alone in postmenopausal women (18,19,80). While postmenopausal estrogen replacement inhibits bone loss, a combined treatment of estrogen and androgen appears to promote bone formation (6). However, the mechanisms through which androgens exert these effects are not well understood. Recent studies have suggested that AR and ER interact, although the consequence of this interaction is unclear (81,82).

To investigate the effect of a disruption of AR in a mouse model of osteoporosis the disclosed AR floxed mice can be used. Osteoporosis can be induced by ovarectomy and the influence of placebo, E2, DHT, or a combined treatment of both steroids on bone morphology and markers of bone turnover can be examined.

(e) Analysis of Bone.

Female mice lacking AR can be ovarectomized at 8 weeks of age. Mice carrying the ACTB-Cre trisgene but with a normal non-floxed AR serve as controls. Ninety days after ovarectomy, mice can be implanted with 60 days release of hormonal pellets for estradiol, DHT, estradiol and DHT, or placebo (Innovative Research of America). After 60 days of treatment, mice from all treatment groups can be sacrificed and the femur and tibia removed. Hisotological examination of bone can be performed as previously described (84). Briefly, bones can be defleshed and fixed in 40% ethanol at 4° C. for 48 hrs. After fixation, bones are embedded in methylmethacrylate without decalcification. Five micon mid-sagittal sections are stained with von Kossa/touluidine blue and toluidine blue at acid pH. Bone formation can be assessed at 15 day intervals after initiation of hormonal treatment by measuring serum osteocalcin levels by RIA (Biomedical Technologies). Bone resorption can also be assessed at 15 day intervals after hormonal treatment by urinary deoxypyridinoline after acid hydroloysis using HPLC (84). Bone histomorphology in terms of cancellous bone volume, osteoblast surface (Ob.S/BS,%), and osteoclast surface (Oc.S/BS,%) is determined as previously described (83).

(9) Alternative Approaches.

As shown herein, mice that are chimeric for the floxed AR have been generated. Alternatively, rather than generating ARKO mice using the ACTB-Cre transgenic mice, the floxed AR mice are crossed to another transgenic line that has general Cre expression, such as EIIa-Cre or CMV-Cre (both commercially available for the Jackson Labs). Removal of exon 2 will interrupt the open reading frame of AR resulting in a truncated AR consisting primarily of the AR N-terminal domain. The mouse Tfm mutation, that results in testicular feminiztion and complete sterility in males, is caused by a point deletion in the N-terminal coding region also resulting in a truncated AR protein consisting of part of the N-terminal domain (8). Therefore, an alternative approach to abolishing AR function via deletion of the mouse AR exon 2 is the use of the mouse Tfm mutation. The Tfm mutation results in a truncated AR and causes complete male sterility due to testicular feminization. The previously described morulae aggregation procedure is used to generate TfmY/XY chimeras (77). These chimeric males are crossed to Tfm/X heterozygous females to yield Tfm/Tfm homozygous females. Previous studies have shown that the Tfm/Tfm females are viable and fertile (77).

(10)Knock Out AR in Specific Tissue

The Cre-lox system has been successfully applied for tissue-specific transgene expression (Orban P C, Chui D, Marth J D. Tissue- and site-specific DNA recombination in transgenic mice. Proc Natl Acad Sci U S A. 1992 Aug 1;89(15):6861-5.), for site specific gene targeting and for exchange of gene sequence by the “knock-in” method (Aguzzi A, Brandner S, Isenmann S, Steinbach J P, Sure U. Transgenic and gene disruption techniques in the study of neurocarcinogenesis. Glia. 1995 Nov;15(3):348-64. Review). Breeding between ARKO male mice and female mice with Cre linked to specific tissue promoters will allow the generation of mice with AR knock-out in a specific tissue.

One of the advantages of creating the floxed AR mice is to provide a base to generate tissue-specific ARKO in selective tissues, such as breast (mating with female MMTV-Cre mice), prostate (mating with female PSA-Cre or probasin-Cre), and liver (mating with female α-fetal protein or albumin-Cre).

(a) Generation of Mice Carrying a Tissue Specific Knockout of AR in the Mammary Gland

AR is expressed in 50-85% of human breast tumors (27,28) and administration of androgens has been found to provide effective adjuvent therapy for breast cancer patients (2,25,26). The polyglutamine repeat length of AR is polymorphic between individuals and short polyglutamine repeats correlates with an increase in AR transcriptional activity in vitro (49,50). Epidemiologically, post-meopausal women who carry AR alleles with short polyglutamine repeat lengths have a decreased risk of breast cancer (87). Conversely, women who inherit BRCA1 germline mutations and carry an AR allele that is less transcriptionally active due to a long polyglutanine repeat have a decreased age of breast cancer onset (51). These observations suggest that AR activity is protective against breast cancer. In vitro observations using breast cancer derived cell lines indicate that androgens, acting through AR, decrease cellular proliferation. DHT inhibits estrogen induced proliferation ZR-75-1, T47-D and MFM-223 cells and this anti-proliferative effect is blocked by the addition of antiandrogens (21-23). However, the mechanism of androgen mediated inhibition of mammary tumor growth is not completely understood.

The effect of both chemically induced and oncogene mediated mammary carcinogenesis in female mice lacking a functional AR in the mammary gland can be examined. DMOA (dimethylbenz(a)anthracene) induced mammary tumors is a well established mouse model of breast cancer. To generate mice with a breast specific disruption of AR for DMBA treatment, the floxed AR strain is crossed to a strain carrying Cre under the control of the whey acidic protein promoter (WAP-Cre) (available from the Jackson Laboratory). WAP-Cre transcription can be activated by pregnancy (88) to generate mice lacking mammary AR. Prior to all tumor induction and progression studies, the AR exon 2 mammary targeted deletion is analyzed to determine the extent of recombination. Wild type mice of the same genetic background are used as controls. Tumor size and growth rate are measured. Mice are euthanized when palpable tumors reach 1.0 cm in diameter and tumor number, size, and location are scored for each mouse. Tumors isolated from AR knockout and control mice can be compared by RNase protection and quantitative PCR for expression of molecular markers associated with mammary carcinomas. Initial screening can be for cathepsin D, p21 (WAF1/CIP1), bc1-2, bc1-x, AIB1, and Her2/Neu. Tumors are also paraffin embedded for morphological analysis, including degree of vascularization. These samples can also be used for immunohistochemical analysis of molecular markers.

To investigate the effect of AR in oncogene induced mammary tumors, a WAP-myc transgenic line that expresses c-myc under the control of the whey acidic protein promoter (89) can be used. The AR floxed mice are crossed with mice homozygous for the WAP-Cre and WAP-myc to generate homozygous AR floxed females carrying WAP-Cre and WAP-Tag transgenes. The WAP promoters can be activated by pregnancy. Tumors are compared to those of AR wild type mice of similar genetic background carrying the WAP-Tag transgene. Tumor latency, number, and growth rate are compared as well as molecular markers as described herein.

After completion of the characterization of the latency and growth rate of tumors induced the tissue specific knockout mice, the effect of androgen and antiestrogen treatment on these animals is determined. AR mammary knockout mice are treated with tamoxifen and DHT, alone or in combination, to determine the effect of hormonal manipulation on the etiology of DMBA or WAP-myc induced mammary tumors.

(i) DMBA-induced Tumorigenesis.

Parous WAP-Cre positive homozygous floxed AR females and wild type AR littermate controls receive six 1 mg weekly doses of DMBA by gastric intubation. Mice do not receive pituitary isografts because the isograft would be expected to interfere with the later hormonal manipulations.

(ii) Hormonal Treatment

The influence of exogenous hormones on DMBA or transgene induced tumors is assessed in female mice that have had two litters (to assure the WAP driven transgenes have been activated). Mice are ovarectomized and implanted with 90 days hormonal release pellets containing placebo, estradiol, DHT, estradiol plus DHT, estradiol with DHT plus flutamide, or estradiol with DHT plus tamoxifen (Innovative Research of America). Tumor latency and growth rate is assessed by measuring tumor size with vernier calipers three times a week until the tumors reach 1.0 cm in size at which time the mouse is sacrificed and the tumor excised for histology as described in Example 2.

(iii) RNase Protection and Quantitative RT-PCR.

Once palpable tumors reach 1.0 cm in diameter, mice are euthanized and tumor number, size, and location are scored for each mouse. RNA is isolated from at least two tumors per mouse by standard methods (90). Tumors not immediately used for RNA extraction are flash frozen in liquid nitrogen and cryopreserved. RNase protection of bc1-2 and bc1-x can be performed using the Pharmingin mAPO-2 kit according to the manufacturer's instructions. Quantitative RT-PCR of cathepsin D, p21(WAF1/CIP1), AIB1 and Her2/Neu can be performed in the presence of a specific synthetic competitor template for each target species that contains a small internal deletion (91). Competitive PCR is performed using a constant amount of cDNA co-amplified with serial dilutions containing a known number of copies of the synthetic competitor. The target cDNA and synthetic competitor are taken to be amplified with the same efficiency due to sequence similarity. The amplification products are separated by gel electrophoresis, visualized by SYBR green (Molecular Dynamics) and quantitated by densitometric scanning using a STORM (Molecular Dynamics).

### 2. Example 2

### Cell Lines for AR Role in Breast and Ovarian Cancers

a) Generation of MCF-7 Cells Lacking Intact AR Loci (MCF-ARKO).

Unlike mouse embryonic stem cells, human tissue culture cells can undergo homologous recombination at high efficiency without the use of isogenic DNA (reviewed in (7)). The method described by Hanson and Sedivy (52) has been used to disrupt the AR loci in the human breast cancer cell line MCF-7. The same method will be used in example 2 to abolish AR expression in additional breast cancer and ovarian cancer cell lines, as well as other AR positive cell lines, to examine the effect on tumorigenesis. As shown in FIG. 1A, a targeting vector was constructed in which a promoterless neomycin cassette has been inserted in frame with the AR ATG. The use of a promoterless selectable marker reduces the number of clones surviving selection that represent random integration events. The 5′ homologous sequence extends 1.1 kb into the human AR 5′ UTP, The 3′ homologous sequence extends 6.2 kb into the AR intron1 (FIG. 1). The flanking sequences were generated by PCR from human LNCaP cells. Established human cell lines have previously been reported to efficiently undergo homologous recombination with non-isogenic DNA (reviewed in (7)). Because some of the cell lines to be targeting for homologous recombination are known to be triploid for the X chromosome, additional targeting vectors were constructed containing hygromycin or zeomycin resistance cassettes in case it is necessary to use multiple rounds of selection to disrupt all AR loci in those lines. The targeting strategy is designed to insert a promoterless selectable marker (with a polyadenylation signal and termination codon) in frame with the AR transcription initiation site. In the homologously recombined locus, transcription from the AR promoter results in the expression of the neomycin cassette and termination of transcription within exon 1, preventing transcription of the remainder of the AR gene. Prior to transfection of the targeting vector into MCF-7 cells, the vector insert verified by DNA sequencing. A G418 resistant clone of MCF-7 was isolated in which the all endogenous AR loci have undergone homologous recombination with the targeting vector and contains no intact AR loci.

b) AR and the Breast Cancer Susceptability Gene BRCA1.

As discussed in the herein, clinical studies suggest that AR may play an inhibitory role in the growth of mammary tumors (1,2,25) Epidemiologically it has been found that women who inherit a mutant BRCA1 allele and an AR allele that has reduced transcriptional activity due to an increased polyglutamine tract have an earlier age of onset of breast cancer than BRCA1 mutation carriers with a more transcriptionally active AR (due to a shorter polyglutamine repeat length) (51). This finding suggests that decreased AR activity, in combination with mutations in other susceptability loci, can enhance mammary tumorigenesis.

c) BRCA1 Enhances AR Mediated Transcription in Breast Cancer Cells.

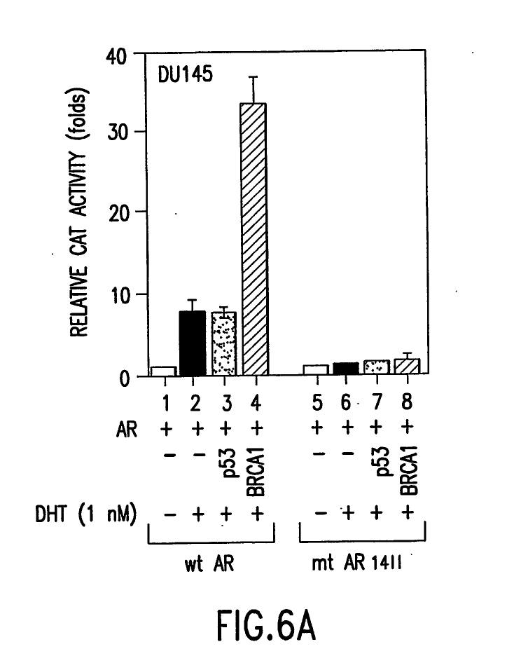

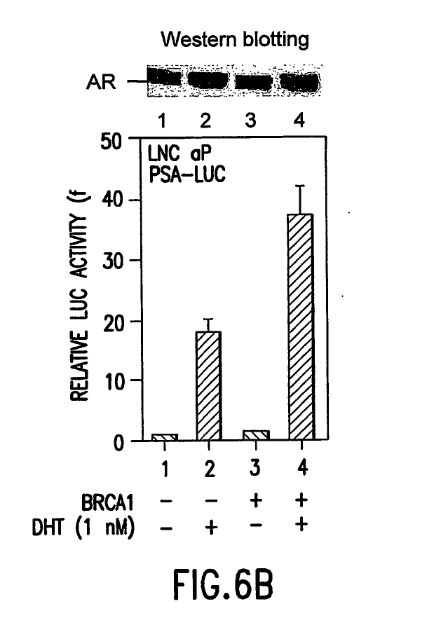

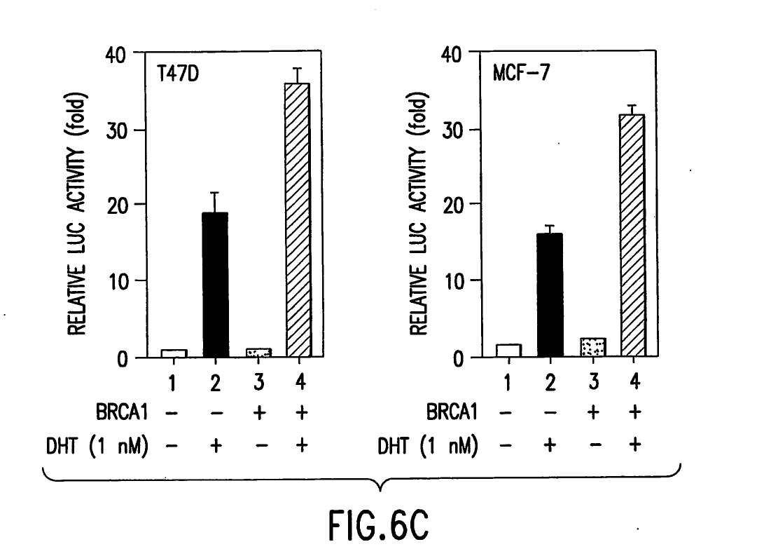

Previously it was determined that AR and BRCA1 physically interact (54). To determine whether BRCA1 influenced AR mediated transcription, AR and BRCA1 were transfected into the AR negativecell line, DU145. As shown in FIG. 6A, BRCA1 enhanced AR transcription of an MMTV-CAT reporter by 4-fold in the presence of DHT. In contrast, p53 had no effect on AR transcription. No reporter gene activity was seen when BRCA1 was co-transfected with the AR (R614H) mutant, which is unable to bind DNA. To establish that the enhanced AR transcription was not due to an increased level of AR protein, BRCA1 was transfected into the AR positive cell line LNCAP. In this cell line, BRCA1 enhanced AR transcription of either MMTV-LUC or PSA-LUC reporters by approximately 2-fold. However, BRCA1 did not alter the AR protein level as shown by Western blot (FIG. 6B). BRCA1 had no effect on either of the reporter constructs tested in the absence of DHT, demonstrating that BRCA1 did not have a transcriptional effect on these reporters independent of AR (FIG. 6B). The transcriptional influence of BRCA1 on AR was then confirmed in the human breast cancer cell lines T47D and MCF-7. As shown in FIG. 6C, cotransfection of AR and BRCA1 resulted in a DHT dependent increase of AR mediated transcription.

d) BRCA1 Functions Synergistically With Other AR Coactivators.

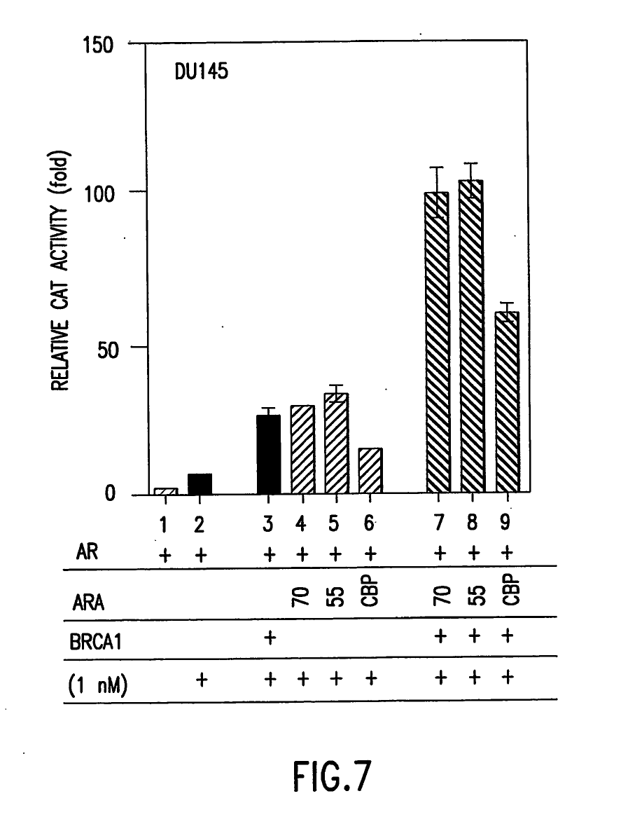

ARA70 and ARA55 are AR coregulators initially identified and characterized by the PI and are capable of enhancing AR transcription by 2 to 10 fold (44,45). As shown in FIG. 7, ARA70N (ARA70 amino acids 1-401), ARA55, CBP, and BRCA1 were able to significantly enhance AR transcription of the MMTV-CAT reporter in DU145 cells. The simultaneous transfection of BRCA1 with these coactivators resulted in a synergistic effect on AR transcription. Previous studies have established that BRCA1 can interact cooperatively with CBP (55,56). Data presented here indicates that BRCA1 also can interact cooperatively with ARA70N and ARA55 to enhance AR mediated transactivation.

e) BRCA1 Enhances AR Transcription of Endogenous Genes in MCF-7 Breast Cancer Cells.

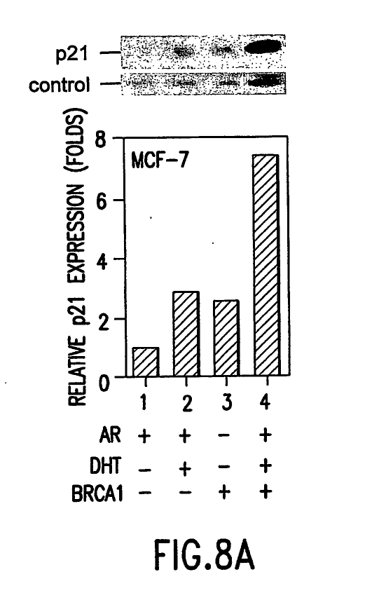

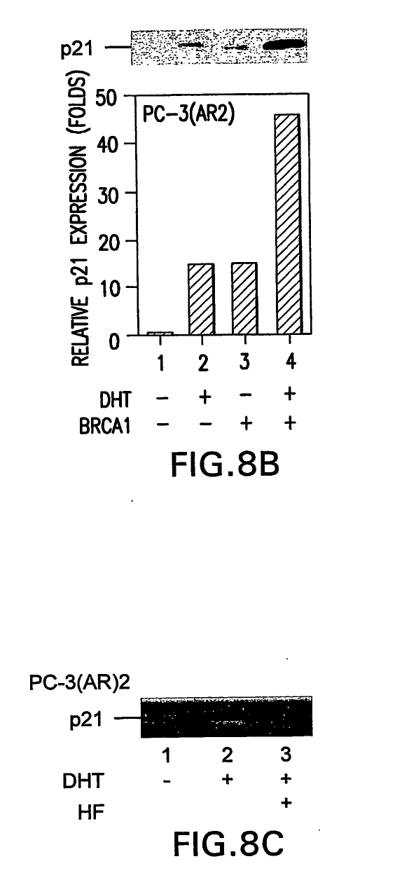

The ability of AR and BRCA1 to regulate the endogenous p21 (WAF1/CIP1) gene in MCF-7 breast cancer cells was examined. As shown in FIG. 8A, BRCA1 and DHT induction of AR independently induced endogenous p21 (WAF1/CIP1) gene expression. This observation was confirmed in the PC-3 (AR2) cell line, a prostate cancer derived cell line that has been stably transfected with AR (57) (FIG. 8B). In agreement with the transient transfection results in FIG. 7, the combination of AR and BRCA1 further enhanced p21 (WAF1/CIP1) expression in response to DHT. In PC-3 (AR2) cells, the antiandrogen hydroxyflutamide blocks the DHT induction of p2l (WAF1/CIP1), suggesting that the effect occurs directly through AR (FIG. 8C).

In summary, BRCA1 functions as an AR coactivator in breast cancer cells by enhancing AR transcription of both transfected reporters and endogenous genes. BRCA1 functions cooperatively with other AR coactivators to enhance AR transactivation suggesting that it may participate in a coregulatory complex with AR. These data therefore clearly demonstrated that AR might play important roles in the breast cancer via interacting with BRCA1.

f) 17β-Estradiol F2) Induces AR Transcriptional Activity in the Presence of ARA70.

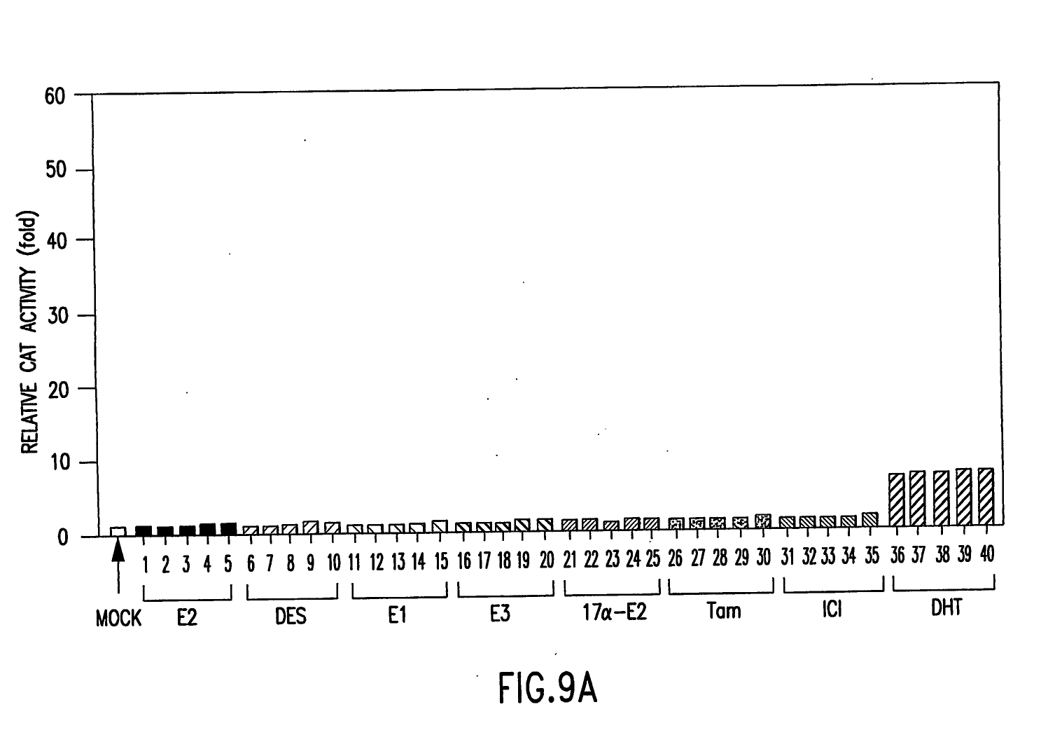

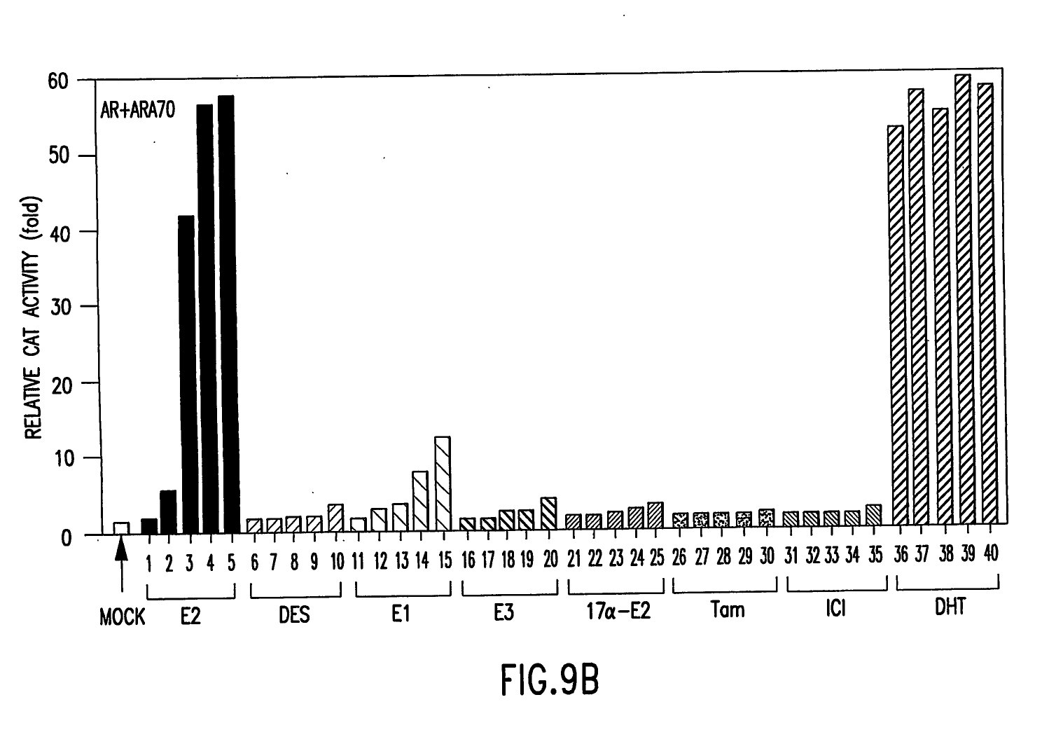

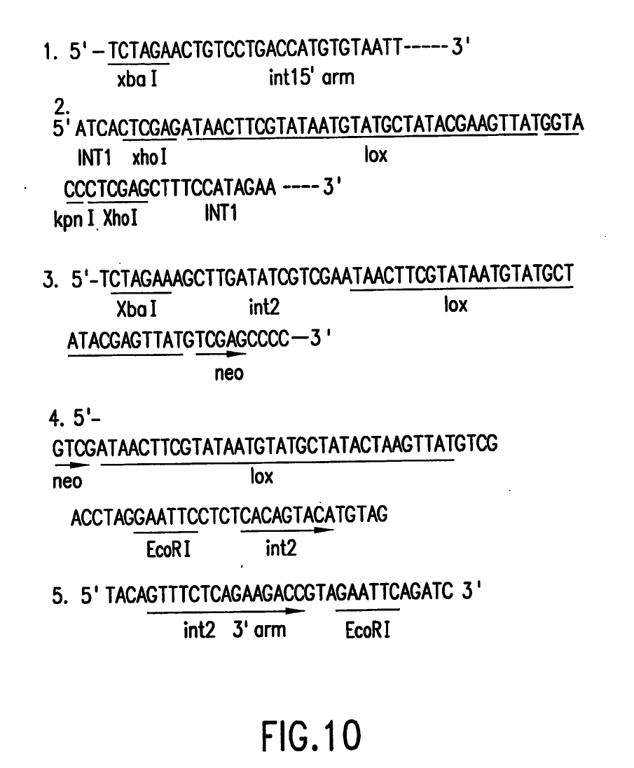

To determine whether E2 can activate AR transcription, the AR and ERα negative cell line DU145 was transfected with AR in the presence of ARA70 or an empty vector control and AR transcriptional activity was monitored using a MMTV-CAT reporter gene. The MMTV promoter does not contain an estrogen response element and therefore E2 is unable to induce ER-mediated transcription of this reporter construct As shown in FIG. 9, AR transcription was induced by 1-10 nM of E2 only in the presence of the AR coregulator ARA70. AR transcription was induced 30-fold by 10 nM E2 in the presence of ARA70, while in the absence of ARA70, E2 did not induce AR transcription. The synthetic estrogen diethylstibesterol (DES) and the estrogen metabolites estrone (E1), estriol (E3), and 17α-estradiol (17α-E2) showed very little induction of AR transcriptional activity in the presence of ARA70 at concentrations up to 1 μM. Likewise, the partial ER agonist tamoxifen and the complete ER antagonist ICI182, 780 (ICI) were unable to induce AR mediated transcription. A similar profile of E2 induction of AR transcription was also found using the prostate specific antigen (PSA) promoter (data not shown). PSA is an androgen target gene that is not transcriptionally activated by the ER (58). These data, together with several other reports (for review, see 59) indicated that in some conditions, E2 could exert its functions via activation of AR.

g) To Generate Human Breast and Ovarian Cell Lines Lacking a Functional AR.

In contrast to mouse embryonic stem cells, human tissue culture cell lines have been shown to undergo efficient homologous recombination with non-isogenic DNA (7). As shown in example 1, MCF-7 cells carrying disrupted AR loci based on the method of Hanson and Sedivy (52) were generated. The targeting construct contains a selectable marker inserted in frame with the AR ATG with the 5′ homologous sequence extending into the UTR and the 3′ homologous sequence extending into intron 1. An analogous strategy has been used by several laboratories to successfully target other loci in human cell lines, including p53 and p21 (WAF1/CIP1) (36,62-64). As depicted in example 1, a targeting vector containing a neomycin resistance marker was generated. This embodiement anticipates that the use multiple rounds of homologous recombination using different selection markers can be required to disrupt the AR genes in the other cell lines to be examined. Targeting vectors identical to the one used in our preliminary results carrying hygromycin and zeomycin selection markers have been constructed. The targeting vectors are used to disrupt the AR loci in ZR-75-1 and T47-D breast cancer cell lines. Three human breast cancer cell lines are targeted to reduce the possibility of artifactual results that might be obtained from using a single cell line. For all assays, the cell line carrying the disrupted AR loci are compared to the parental cell line.

Once cells from each parental cell line have been isolated lacking intact AR loci as determined by Southern blot analysis, Western blots can be performed to confirm the absence of AR protein. Once the AR negative cell lines (MCF-ARKO, ZR-ARKO, and T47-ARKO) have been established, they are compared to the parental, AR positive cell lines in terms of the ability of androgens to inhibit estradiol (E2) induced proliferation and down regulate bc1-2 expression (23,31). The ARKO breast cancer cells can be unresponsive to androgen and can continue to proliferate in the presence of E2 as well as E2 plus DHT. Alternatively, E2 can go through AR to modulate cell growth, then lacking of AR can influence the E2 effect on the cell growth. The antiandrogen flutamide is used to block DHT-mediated AR activation. The steroidal treatment regime for each parental and ARKO breast cancer cell line for comparison of cellular proliferation is detailed in Table 3 below.

TABLE 3
Steroidal Treatment of Breast Cancer Cell Lines

| Cell Line | Vehicle | DHT | E2 | Flutamide |
|---|---|---|---|---|
| Parent | + | − | − | − |
| Cells;(MCF-7 | | | | |
| T47-D | − | + | − | − |
| Or ZR-75-1) | − | − | + | − |
| | − | − | − | + |
| | − | + | − | + |
| | − | + | + | − |
| | − | + | + | + |
| Knock-out | + | − | − | − |
| Cells:(MCF- | | | | |
| ARKO | | | | |
| T47-ARKO | − | + | − | − |
| Or ZR-ARKO) | − | − | + | − |
| | − | − | − | + |
| | − | + | − | + |
| | − | + | + | − |
| | − | + | + | + |

The targeting vector used for targeted disruption of AR in MCF-7 cells is also used to disrupt the AR loci in the human ovarian carcinoma cell lines OVCAR-3, ES-2, and SKOV-3. The strategy for generation of ovarian ARKO cell lines will be the same as described above for the breast cancer cell lines. The successful homologous recombination and disruption of the AR gene in the ovarian cell lines can be determined by Southern blot. Once all AR loci have been determined to be disrupted in a cell line (ARKO), Western blots can be used to confirm that AR is not expressed in the ARKO lines. The parental cell lines OVCAR-3, ES-2, and SKOV-3 can be compared to their associated AR negative cell lines OVCAR-ARKO, ES-ARKO, and SKOV-ARKO in all assays. Initial characterization of the ARKO cell lines with their respective parental cell lines can be of the influence of androgens on cellular proliferation. It has been suggested that progesterone may contribute to ovarian cancer risk (61) and therefore progesterone as well as estrogen and androgen are examined. Table 4 below summarizes the hormonal treatments of the ovarian ARKO and parental cell lines.

TABLE 4
Steroidal Treatment of Ovarian Cell Lines

| Cell Line | Vehicle | DHT | E2 | Progesterone | Flutamide |
|---|---|---|---|---|---|
| Parent | + | − | − | − | − |
| Cells:(OVCAR-3 | − | + | − | − | − |
| SVOV-3 | − | − | + | − | − |
| ES-2) | − | − | − | + | − |
| | − | − | − | − | + |
| | − | − | + | + | − |
| | − | + | + | − | − |
| | − | + | + | − | + |
| | − | + | − | + | − |
| | − | + | − | + | + |
| | − | + | + | + | − |
| | − | + | + | + | + |
| Knock-out Cells: | + | − | − | − | − |
| (OVCAR-ARKO, | − | + | − | − | − |
| SVOV-ARKO, | − | − | + | − | − |
| Or ES-ARKO) | − | − | − | + | − |
| | − | − | − | − | + |
| | − | − | + | + | − |
| | − | + | + | − | − |
| | − | + | + | − | + |
| | − | + | − | + | − |
| | − | + | − | + | + |
| | − | + | + | + | − |
| | − | + | + | + | + |

h) Selection Conditions.

To generate cell lines lacking a functional AR gene, we will follow the procedure of Hanson and Sedivy (4). MCF-7 cells have already been targeted (example 1). The targeting vectors are electroporated into ZR-75-1, T47-D, ES-2, SCOV-3 and OVCAR-3 cells. Electroporated cells are maintained for 48 h in the absence of selection before being transferred to the appropriate selective media. Because ZR-75-1 cells have been reported to have three X chromosomes (67) and OVCAR-3 cells are near triploid, it is anticipated that three selectable markers are required to disrupt all copies of the AR gene present in both cell lines. The first round of targeting is performed using a neomycin marker and recombinants are selected in G418 containing medium. Surviving colonies are cloned and homologous recombinants can be identified by Southern blot. Homologous recombinants isolated in this targeting round can be subjected to electroporation with a targeting vector containing a hygromycin resistance marker. After recovery, cells are selected in media containing hygromycin B and G418. Cells surviving in the selective media are cloned and homologous recombinants can be identified by Southern blot. A final round of targeting is performed using a zeomycin resistance marker. Southern blot analysis can enable us to confirm that all copies of AR have been targeted. Other selectable markers can be used at any recombination step.

i) Cellular Proliferation.

Equal numbers of ARKO and the associated parental cells are plated in 60 mm dishes in phenol red free RPMI 1640 media containing 5% charcoal-dextran stripped FBS. Cells are treated with vehicle, 1 nM estradiol, 10 nM DHT, 3 μM flutamide, 10 nM DHT in the presence of 3 μM flutamide, 1 nM estradiol with 10 nM DHT, 1 nM estradiol with 3 μM flutamide, and 1 nM estradiol with 10 nM DHT and 3 μM flutamide. Cell number are monitored for 24 days and determined by MTT assay. For the ovarian cell lines, the ovarian ARKO and parental cell lines are analyzed with the additional treatments of 1 nM progesterone, 1 nM progesterone with 10 nM DHT, and 1 nM progesterone with 10 nM DHT and 3 μM flutamide. The treatment groups are summarized in Tables 3 and 4 above. Ovarian cancer cells are examined for the relative effect of progesterone and androgens either singly or in combination.

j) Immunoblotting for bc1-2.

Breast and ovarian ARKO cells are treated for 10 days with the hormonal regimes described above. The associated parental cell line for each ARKO cell line is used as a control. Cells are lysed in 50 mM Tris-HCl/pH7.5, 0.25 M NaCl, 10% (v/v) Triton X-100, 0.1% (w/v) SDS, 0.5% (w/v) deoxycholate, 1 mM EDTA, 0.1 mM PMSF, and 1 μg/ml aprotinin. 15 μg of protein extract are separated on 12% SDS-PAGE gels and electroblotted onto nitrocellulose. Blots are probed with a bc1-2 reactive antibody (Santa Cruz) and visualized using ECL-based detection (Amersham).

k) To Examine the Tumorigenicity of Human Breast Cancer Cells Carrying a Targeted Disruption of AR.

Several observations indicate that androgens can be inhibitors of breast cancer proliferation. Estrogen induced proliferation of the AR positive breast cancer cell lines ZR-75-1 and T47-D can be inhibited by DHT (21,23) and this anti-proliferative effect can be reverse by treatment with anti-androgens. E2 induced proliferation of ZR-75-1 cells in ovarectomized nude mice is inhibited by DHT (4). Additionally, E2 enhanced growth of DMBA induced mammary tumors in ovarectomized rats is inhibited by DHT treatment, and this inhibition is reversed by administration of the anti-androgen flutamide (3). In both pre- and postmenopausal breast cancer patients, androgens or androgenic compounds such as testosterone propionate (1), fluoxymesterone (2), or calusterone (25,26) have been found to be effective adjuvant therapies. However, the mechanism by which androgens modulate breast cancer growth is not well understood.

After the initial in vitro characterization of the ARKO cells, mouse xenograft experiments are performed. The ARKO cells are subcutaneously injected into ovarectomized nude mice and the parental cell lines serve as xenograft controls. This experiment allows for the comparison of AR positive and AR negative breast carcinoma cells in animals that carry a functional AR gene without the potential confounding effects of using differently derived tumor cell lines. Tumor growth rate is compared in ARKO and parental cell line recipients in response to estradiol treatment, DHT treatment, or combined administration of estradiol and DHT. The affect of antiestrogens (e.g. tamoxifen) or antiandrogens (e.g. hydroxyflutamide) on tumor growth is investigated. It is anticipated by this embodiement that a combined treatment of DHT and antiestrogen can have the greatest inhibitory effect on parental derived tumors (4) while ARKO derived tumors can respond only to antiestrogens (4). In addition to growth rate, tumors are examined histologically to determine the morphology and degree of vascularization between ARKO cell tumors and tumors generated by the parental cell lines under the different hormonal regimes.

1) To Examine the Tumorigenicity of Human Ovarian Cancer Cells Carrying a Targeted Disruption of AR.

In contrast to breast cancer, epidemiological evidence and animal studies suggest that androgens may enhance the risk of ovarian cancer. In a prospective cohort study, prediagnostic serum androgens were found to be approximately 50% higher in case subjects (5). The AR coactivator ARA70 is overexpressed in 85% of ovarian carcinomas, suggesting that enhancement of AR transcription by ARA70 may be involved in ovarian carcinogeneisis (44,65). In the guinea pig, long term administration of androgens, but not estrone, induces ovarian epithelial cyst formation (66). Neonatally thymodectamized mice develop dysgenic ovaries and tubular adenomas. Prior to tumor formation, the ovaries of these mice produce elevated levels of androstrenedione and testosterone, but not estrogens (68). AR is expressed in the ovary, particularly in granulosa cells (69,70), and androgens acting through AR have been implicated in ovarian function (71,72).

The targeting vector used for targeted disruption of AR in MCF-7 cells is used to disrupt the AR loci in the human ovarian carcinoma cell lines OVCAR-3, ES-2, and SKOV-3. Three cell lines are examined to reduce the possibility of experimental artifacts that might arise by analysis of only one line. The targeting construct contains the 1.1 kb of the AR 5′ UTR, a neomycin marker in frame with the AR ATG, and 6.2 kb of the AR intron 1 sequence. Analogous constructs have also been generated in which the neomycin resistance marker has been substituted for hygromycin and zeomycin. These constructs will be used to disrupt the AR genes in the ovarian cell lines. This embodiement anticipates that multiple rounds of targeting using different selectable markers can be required to disrupt all of the AR loci in some of these cell lines. The successful homologous recombination and disruption of the AR gene in the ovarian cell lines can be determined by Southern. Once all AR loci are determined to be disrupted in a cell line (ARKO), Western blots can be used to confirm that AR is not expressed in the ARKO lines. The parental cell lines OVCAR-3, ES-2, and SKOV-3 are be compared to their associated AR negative cell lines OVCAR-ARKO, ES-ARKO, and SKOV-ARKO in all assays. Initial characterization of the ARKO cell lines with their respective parental cell lines can be of the influence of androgens on cellular proliferation. Cells are treated with vehicle, estrogen, progesterone, or androgen alone, and with estrogen+androgen or progesterone+androgen. The ARKO and parental cell lines are then used in mouse xenograft experiments to determine their tumorigenic capacity in vivo. Recipients of ARKO or parental cell lines will be treated with vehicle, estradiol, progesterone, or DHT alone or in combination. All tumors are analyzed for growth rate, morphology, and degree of vascularization under the different hormonal regimes.

m) Nude Mouse Xenografts.

Female homozygous nude mice (obtained from the Jackson Laboratory) are ovarectomized. One week after ovarectomy, all mice (except sham operated controls) are implanted with 1.7 mg 90 day release 17β-estradiol hormonal pellets or placebo pellets (Innovative Research of America, Florida). Ovarectomized mice are also implanted with 1.5 mg 90 day release progesterone hormonal tablets. Only the ovarian cancer based cell lines will be examined in progesterone treated animals. Animals will be subcutaneously injected with 2×106 of the ARKO or parental cells at the time of hormonal implant Hormonal pellets will be replaced as required. Mice are used for further study when the tumor size reaches 0.5 cm. At this point, the estradiol releasing pellet will be replaced with 90 day release pellets containing 0.72 mg of estradiol to release a more physiological dose of estrogen. Mice to be treated with estradiol and DHT will also receive 90 day release hormonal pellets containing 12.5 mg of DHT (Innovative Research of America, Florida). Tamoxifen can be administered as 5 mg 90 day release hormonal pellets and flutamide can be administered as a 25 mg 90 day release pellet (Innovative Research of America, Florida). Tumor size can be measured three times a week using Vernier calipers over a period of 50 days. After 50 days, animals can be sacrificed and the tumors excised for paraffin embedding and histological analysis to determine if there are morphological differences between AR positive and AR negative tumors, for example, in the degree of vascularization.

The ZR-ARKO derived tumors are expected to continue estradiol induced proliferation in the presence of DHT, while DHT is expected to inhibit the estradiol-mediated proliferation of ZR-75-1 tumors as previously reported (4). A combined treatment of DHT and antiestrogen is expected to have the greatest inhibitory effect on ZR-75-1 derived tumors (4) while ZR-ARKO derived tumors are expected to respond only to antiestrogens (4).

AR is also targeted T47-D cells using the methods described above. The T47D cell line is hypotriploid, and clones carrying two X chromosomes are isolated. The AR locus can then be targeted using neomycin and hygromycin resistance markers as described above.

### 3. Example 3

### Generation and Characterization of Androgen Receptor Knock Out (ARKO) Mice: An In Vivo Model for the Study of Androgen Functions in Selective Tissues

The increasing evidence shows that the androgen and AR may play important roles both in male and female physiological processes. The testicular feminization/y mice (Tfm) and the patients of androgen insensitive syndrome (AIS) are the natural models for the study of the loss of androgen function in male. However, there has been a lack of female models and the classical knock-out strategy does not work because AR is located on the X chromosome, which is critical for male fertility. To generate female AR knock-out mice, conditional knockout model, such as a cre-lox strategy can be used. The cre-lox system utilizes the expression of P1 phage Cre recombinase to catalyze the excision of DNA located between flanking lox site. This strategy differs from the standard targeted disruption procedure in that ES cells are generated in which the targeted segment is not disrupted but flanked by lox sites (floxed). The target gene thus functions normally and mice can be bred to homozygosity for the targeted locus. Disclosed herein is the generation and characterization of an AR knock out (ARKO) in female and male mice. The disclosed mice show that the bone density of male KO mice is reduced due to the higher increases of osteclast than osteoblast. In addition, the female AR knock mice have lower fertility due to disruption of ovaulation. With the floxed AR mice, it is possible to create tissue specific and inducible ARKOs for specical functional studies.

Androgen receptor (AR), a member of the nuclear receptor superfamily was first cloned in 1988 (Chang C S, Kokontis J, Liao S T. Molecular cloning of human and rat complementary DNA encoding androgen receptors. Science. 1988 Apr 15;240(4850):324-6; Chang C S, Kokontis J, Liao S T. Structural analysis of complementary DNA and amino acid sequences of human and rat androgen receptors. Proc Natl Acad Sci USA. 1988 Oct;85(19):7211-5; and Lubahn D B, Joseph D R, Sullivan P M, Willard H F, French F S, Wilson E M. Cloning of human androgen receptor complementary DNA and localization to the X chromosome. Science. 1988 Apr 15;240(4850):327-30.). It contains a N-terminal transactivation domain, a central DNA binding domain (DBD), and a C-terminal ligand binding domain (LBD) (Chang C, Saltzman A, Yeh S, Young W, Keller E, Lee H J, Wang C, Mizokami A. Androgen receptor: an overview. Crit Rev Eukaryot Gene Expr. 1995;5(2):97-125. Review.). AR may form a dimer and interact with many coregulators to modulate androgen target genes (Heinlein C A, Chang C. Androgen Receptor (AR) Coregulators: An Overview. Endocrine Review 2002; 23, 175-200). In addition to its natural ligands, Testosterone (I) and Dihydrotestosterone (DHT), 17β-estradiol (E2) can also induce AR transactivation in the presence of some selective coregulators in some selective tissues (Yeh S, Miyamoto H, Shima H, Chang C. From estrogen to androgen receptor: a new pathway for sex hormones in prostate. Proc Natl Acad Sci USA. 1998 May 12;95(10):5527-32.). The increasing evidence shows that the androgen and AR may also play important roles in female physiological processes, including folliculogenesis (Donath J, Michna H, Nishino Y. The antiovulatory effect of the antiprogestin onapristone could be related to down-regulation of intraovarian progesterone (receptors). J Steroid Biochem Mol Biol. 1997 May;62(1):107-18.), the bone metabolism (Compston J E. Sex steroids and bone. Physiol Rev. 2001 Jan;81(1):419-447. Review.), auto-immune diseases (Olsen N J, Kovacs W J. Effects of androgens on T and B lymphocyte development. Immunol Res. 2001;23(2-3):281-8. Review), the maintanence of brain functions (Poletti A, Martini L. Androgen-activating enzymes in the central nervous system. J Steroid Biochem Mol Biol. 1999 Apr-Jun;69(1-6):117-22. Review.) and several female cancers of the breast, ovary and endometrium (Liao D J, Dickson R B. Roles of androgens in the development, growth, and carcinogenesis of the mammary gland. J Steroid Biochem Mol Biol. 2002 Feb;80(2):175-89; Wang P H, Chao H T, Liu R S, Cho Y H, Ng H T, Yuan C C. Diagnosis and localization of testosterone-producing ovarian tumors: imaging or biochemical evaluation. Gynecol Oncol. 2001 Dec;83(3):596-8; and Sasakj M, Dahiya R, Fujimoto S, Ishikawa M, Oshimura M. The expansion of the CAG repeat in exon 1 of the human androgen receptor gene is associated with uterine endometrial carcinoma. Mol Carcinog. 2000 Mar;27(3):2374.)

Androgens are the most conspicuous of the steroid hormones in ovary. The concentrations of T and E2 in the late-follicular phase, when estrogens are at their peak, are 0.06-0.10 mg/day and 0.04-0.08 mg/day respectively (Risch H A. Hormonal etiology of epithelial ovarian cancer, with a hypothesis concerning the role of androgens and progesterone. J Natl Cancer Inst. 1998 Dec 2; 90(23):1774-86. Review.). The ratio of androgens to estrogens in the ovarian veins of postmenopausal women is 15 to 1 (Risch H A. Hormonal etiology of epithelial ovarian cancer, with a hypothesis concerning the role of androgens and progesterone. J Natl Cancer Inst. 1998 Dec 2; 90(23):1774-86. Review; Doldi N, Belvisi L, Bassan M, Fusi F M, Ferrari A. Premature ovarian failure: steroid synthesis and autoimmunity. Gynecol Endocrinol. 1998 Feb;12(1):23-8.Endocrinol. 1998 Feb;12(1):23-8.). AR is expressed predominantly in the granulosa cells of the ovary. With the overproduction of ovarian androgen, women with polycystic ovarian syndrome suffer from impairment of ovulatory function, which is characterized by the increasing number of small antral follicles, but an arrest in grafian follicles development (Futterweit W, Deligdisch L. Effects of androgens on the ovary. Fertil Steril. 1986 Aug;46(2):343-5; Fauser B C, Pache T D, Lamberts S W, Hop W C, de Jong F H, Dahl K D. Serum bioactive and immunoreactive luteinizing hormone and follicle-stimulating hormone levels in women with cycle abnormalities, with or without polycystic ovarian disease. J Clin Endocrinol Metab. 1991 Oct;73(4):811-7.). These symptoms suggest that AR may play a proliferative role in early folliculogenesis but change to an inhibitory role in late folliculogenesis. The recent studies conducted in animals also supported this hypothesis (Harlow C R, Shaw H J, Hillier S G, Hodges J K. Factors influencing follicle-stimulating hormone-responsive steroidogenesis in marmoset granulosa cells: effects of androgens and the stage of follicular maturity. Endocrinology. 1988 Jun; 122(6):2780-7; Weil S, Vendola K, Zhou J, Bondy C A. Androgen and follicle-stimulating hormone interactions in primate ovarian follicle development. J Clin Endocrinol Metab. 1999 Aug;84(8):2951-6.). Administration of DHT in rhesus monkeys has increased the number of primary, preantral and small antral follicles. Since DHT is the metabolite of T and cannot be aromatized into E2, these data may suggest that the proliferative effect might go through the DHT-AR and not the E2-ER pathways. (Vendola K, Zhou J, Wang J, Famuyiwa O A, Bievre M, Bondy C A. Androgens promote oocyte insulin-like growth factor I expression and initiation of follicle development in the primate ovary. Biol Reprod. 1999 Aug;61(2):353-7.).

In the cartilage and bone system, AR is expressed in chondrocytes, osteoblasts, osteocytes (Benz D J, Haussler M R, Thomas M A, Speelman B, Komm B S. High-affinity androgen binding and androgenic regulation of alpha 1(I)-procollagen and transforming growth factor-beta steady state messenger ribonucleic acid levels in human osteoblast-like osteosarcoma cells. Endocrinology. 1991 Jun;128(6):2723-30.), and in osteoclasts (Mizuno Y, Hosoi T, Inoue S, Ikegami A, Kaneki M, Akedo Y, Nakamura T, Ouchi Y, Chang C, Orimo H. Immunocytochemical identification of androgen receptor in mouse osteoclast-like multinucleated cells. Calcif Tissue Int. 1994 Apr,54(4):325-6.). Clinical studies suggested that combined therapy of estrogens plus androgens may enhance bone mineral density and bone mass to a more significant degree than estrogen therapy alone in postmenopausal women (Davis S R, McCloud P, Strauss B J, Burger H. Testosterone enhances estradiol's effects on postmenopausal bone density and sexuality. Maturitas. 1995 Apr;21(3):227-36; Castelo-Branco C, Vicente J J, Figueras F, Sanjuan A, Martinez de Osaba M J, Casals E, Pons F, Balasch J, Vanrell J A. Comparative effects of estrogens plus androgens and tibolone on bone, lipid pattern and sexuality in postmenopausal women. Maturitas. 2000 Feb 15;34(2):161-8.). However, the mechanism of androgen action on bone system remains controversial. Some studies suggest that the effect is mainly through the aromatase to transform the androgen to estrogen (Schweikert H U, Rulf W, Niederle N, Schafer H E, Keck E, Kruck F. Testosterone metabolism in human bone. Acta Endocrinol (Copenh). 1980 Oct;95(2):258-64.). Other studies show that administration of antiandrogens, including flutamide and Casodex, to female mice resulted in osteopenia and could not be reversed by aromatase inhibitors suggesting the direct role of AR in bone metabolism (Lea C K, Flanagan A M. Ovarian androgens protect against bone loss in rats made oestrogen deficient by treatment with ICI 182,780. J Endocrinol. 1999 Jan; 160(1):111-7; Migliaccio A, Castoria G, Di Domenico M, de Falco A, Bilancio A, Lombardi M, Barone M V, Ametrano D, Zannini M S, Abbondanza C, Auricchio F. Steroid-induced androgen receptor-oestradiol receptor beta-Src complex triggers prostate cancer cell proliferation. EMBO J. 2000 Oct 16;19(20):5406-17; and Panet-Raymond V, Gottlieb B, Beitel L K, Pinsky L, Trifiro M A. Interactions between androgen and estrogen receptors and the effects on their transactivational properties. Mol Cell Endocrinol. 2000 Sep 25;167(1-2): 139-50.).

For AR, the testicular feminization/y mice (Tfm) and the patients of androgen insensitive syndrome (AIS) are the natural models for the study of the loss of androgen function in male (Soule S G, Conway G, Prelevic G M, Prentice M, Ginsburg J, Jacobs H S. Osteopenia as a feature of the androgen insensitivity syndrome. Clin Endocrinol (Oxf). 1995 Dec;43(6):671-5.). However, there has been a lack of female models and the classical knock-out strategy does not work because AR is located in X chromosome, which is critical for male fertility. To generate female AR knock-out mice, a conditional knockout strategy, such as a cre-lox strategy,can be used. The cre-lox system utilizes the expression of P1 phage Cre recombinase to catalyze the excision of DNA located between flanking lox site (Holt C L, May G S. A novel phage lambda replacement Cre-lox vector that has automatic subcloning capabilities. Gene. 1993 Oct 29;133(1):95-7.). This strategy differs from the standard targeted disruption procedure in that ES cells are generated in which the targeted segment is not disrupted but flanked by lox sites (floxed). The target gene thus functions normally and mice can be bred to homozygosity for the targeted locus. Here we describe the generation and characterization of AR knock outs (ARKO) in female and male mice.

G. References

1. Group, C. B. C. (1964) J. Amer. Med. Assoc. 188, 1069-1072

2. Torney, D. C., Lippman, M. E., Edwards, B. K., and Cassidy, J. G. (1983) Annals of Internal Medicine 98, 139-144

3. Dauvois, S., Li, S., Martel, C., and Labrie, F. (1989) Breast Cancer Research and Treatment 14, 299-306

4. Dauvois, S., Geng, C. -S., Levesque, C., Merand, Y., and Labrie, F. (1991) Cancer Research 51, 3131-3135

5. Helzouer, K. J., Alberg, A. J., Gordon, G. B., Longcope, C., Bush, T. L., Hoffman, S. C., and Comstock, G. W. (1995) JAMA 274, 1926-1930

6. Rosenberg, M. J., King, T. D. N., and Timmons, M. C. (1997) J. Reprod. Med. 42, 394-404

7. Sedivy, J. M., Vogelstein, B., Liber, H. L., Hendrickson, E. A., and Rosmarin, A. (1999) Science 283, 9

8. Gaspar, M. -L., Meo, T., Bourgarel, P., Guenet, J. -L., and Tosi, M. (1991) Proc. Natl. Acad. Sci. USA 88, 8606-8610

9. Quigley, C. A., De Bellis, A., Marschke, K. B., El-Awady, M. K., Wilson, E. M., and French, F. S. (1995) Endocr. Rev. 16, 271-321

10. Vanderschueren, D., and Bouillon, R. (1995) Calcif. Tissue Int. 56, 341-346

11. Mizuno, Y., Hosoi, T., Inoue, S., Ikegami, A., Kaneki, M., Akedo, Y., Nakamura, T., Ouchi, Y., Chang, C., and Orimo, H. (1994) Calcif. Tissue Int. 54, 325-326

12. Jassel, S. K., Barrett-Connor, E., and Edelstein, S. L. (1995) J. Bone. Miner. Res. 10, 650-654

13. Murphy, S., Khaw, K., Cassidy, S., and al, e. (1993) Bone Miner. 20, 133-140

14. Steinberg, K. K., Frenitetulaer, L. W., DePuey, E. G., and al, e. (1988) J. Clin. Endocrinol. Metab. 69, 533-539

15. Devogelaer, J. P., DeCooman, S., and deDeuxxhaisnes, C. N. (1992) Maturitas 15, 17-23

16. Vermeulen, A. (1975) J. Clin. Endocrinol. Metab. 42, 247-253

17. Raisz, L. G., Wiita, B., Artis, A., Bowen, A., Schwartz, S., Trahiotis, M., Shoukri, K., and Smith, J. (1996) J. Clin. Endocrinol. Metab. 81, 37-43

18. Watts, N. B., Notelovitz, M., Timmons, M. C., Addison, W. A., Wiita, B., and Downey, L. J. (1995) Obstet. Gynacol. 85, 529-537

19. Castelo-Branco, C., Vicente, J. J., Figueras, F., Sanjuan, A., Martinez de Osaba, M. J., Casals, E., Pons, F., Balasch, J., and Vanrell, J. A. (2000) Maturitas 34, 161-168

20. Xie, B., Tsao, S. W., and Wong, Y. C. (1999) Carcinogenesis 20, 1597-1606

21. Birrell, S. N., Bentel, J. M., Hickey, T. E., Ricciardelli, C., Weger, M. A., Horsfall, D. J., and Tilley, W. D. (1995) J. Steroid Biochem. Molec. Biol. 52, 459-467

22. Hackenberg, R., and Schulz, K. -D. (1996) J. Steroid Biochem. Molec. Biol. 56, 113-117

23. Poulin, R., Baker, D., and Labrie, F. (1988) Breast Cancer Res. Treat. 12, 213-225

24. Szelei, J., Jimenez, J, Soto, A. M., Luizzi, M. F., and Sonnenschein, C. (1997) Endocrinology 138, 1406-1412

25. Gordan, G. S., Halden, A., Horn, Y., Fuery, J. J., Parsons, R. J., and Walter, R M. (1973) Oncology 28, 138-146

26. Goldenberg, I. S., Waters, M. N., Ravdin, R. S., Ansfield, F. J., and Segaloff, A. (1973) JAMA 223, 1267-1268

27. Soreide, J. A., Lea, O. A., Varhjaug, J. E., Skarstein, A., and Kvinnsland, S. (1992) Eur. J. Surg. Oncol. 18, 112-118

28. Lea, O. A., Kvinnsland, S., and Thorsen, T. (1989) Cancer Research 49, 7162-7167

29. Poulin, R., Simard, J., Labrie, C., Petitclerc, L., Dumont, M., Lagace, L., and Labrie, F. (1989) Endocrinology 125, 392-399

30. Couture, P., Theriault, C., Simard, J., and Labrie, F. (1993) Endocrinology 132, 179-185

31. Lapointe, J., Fournier, A., Richard, V., and Labrie, C. (1999) Endocrinology 140, 416-421

32. Kroemer, G. (1997) Nat. Med. 3, 614-620

33. Lu, S., Liu, M., Epner, D. E., Tsai, S. Y., and Tsai, M. -J. (1999) Mol. Endocrinol. 13, 376-384

34. Brugarolas, J., Chandrasekaran, C., Gordon, J. I., Beach, D., Jacks, T., and Hannon, G. J. (1995) Nature 377, 552-557

35. Halevy, O., Novitch, B. G., Spicer, D. B., Skapek, S. X., Rhee, J., Hannon, G. J., Beach, D., and Lassar, A. B. (1995) Science 267, 1018-1021

36. Waldman, T., Kinzler, K. W., and Volgelstein, B. (1995) Cancer Research 55, 5187-5190

37. Easton, D. F., Bishop, D. T., Ford, D., Crockford, G. P., and Consortium, T. B. C. L. (1993) Am. J. Hum. Genet. 52, 678-701

38. Cornelisse, C. J., Comelis, R. S., and Devilee, P. (1996) Path. Res. Pract. 192, 684-693

39. Tirkkonen, M., Johannsson, O., Agnarsson, B. A., Olsson, H., Ingvarsson, S., Karhu, R., Tanner, M., Isola, J., Barkardottir, R. B., Borg, A., and Kalhoniemi, O. P. (1997) Cancer Res. 57, 1222-1227

40. Scully, R., Chen, J., Plug, A., Xiao, Y., Weaver, D., Feunteun, J., Ashley, T., and Livingston, D. M. (1997) Cell 88, 265-275

41. Somasundaram, K., Zhang, K., Zeng, Y. X., Houvras, Y., Peng, Y., Wu, G. S., Licht, J. D., Weber, B. L., and El-Deiry, W. S. (1997) Nature 389, 187-190

42. Chen, Y., Farmer, A. A., Chen, C. F., Jones, D. C., Chen, P. L., and Lee, W. H. (1996) Cancer Res. 56, 3168-3172

43. Vaughn, J. P., Davis, P. L., Jarboe, M. D., Huper, G., Evans, A. C., Wiseman, R. W., Berchuck, A., Iglehart, J. D., Futreal, P. A., and Marks, J. R. (1996) Cell Growth Diffr. 7, 711-715

44. Yeh, S., and Chang, C. (1996) Proc. Natl. Acad. Sci. U.S.A 93, 5517-5521

45. Fujimoto, N., Yeh, S., Kang, H., Inui, S., Chang, H. C., Mizokami, A., and Chang, C. (1999) J. Biol. Chem. 274, 8316-8321

46. Kang, H. -Y., Yeh, S., Fujimoto, N., and Chang, C. (1999) J. Biol. Chem. 274, 8570-8576

47. Hsiao, P. W., and Chang, C. (1999) J. Biol. Chem. 274, 22373-22379

48. Hsiao, P. W., Lin, D., Nakao, R., and Chang, C. (1999) J. Biol. Chem. 274, 20229-20234

49. Gao, T., Marcelli, M., and McPhaul, M. J. (1996) Journal of Steroid Biochemisty and Molecular Biology 59, 9-20

50. Tut, T. G., Ghadessy, F. J., Trifiro, M. A., Pinsky, L., and Yong, E. L. (1997) J. Clin. Endocinol. Metab. 82, 3777-3782

51. Rebbeck, T. R., Kantoff, P. W., Krithivas, K., Neuhausen, S., Blackwood, M. A., Godwin, A. K., Daly, M. B., Narod, S. A., Garber, J. E., Lynch, H. T., Weber, B. L., and Brown, M. (1999) Am. J. Hum. Genet. 64, 1371-1377

52. Hanson, K. D., and Sedivy, J. M. (1995) Molecular and Cellular Biology 15, 45-51

53. Gu, H., Zou, Y. R., and Rajewsky, K. (1993) Cell 73, 1155-1164

54. Yeh, S., Hu, Y. C., Rahman, M., Lin, H. K., Hsu, C. L., Ting, H. J., Kang, H. Y., and Chang, C. (2000) Proc. Natl. Acad. Sci. USA 97, 11256-11261

55. Cui, J. Q., Wang, H., Reddy, E. S., and Rao, V. N. (1998) Oncol. Rep. 5, 585-589

56. Welcsh, P. L., Schubert, E. L., and King, M. -C. (1998) Clin. Genet. 54, 447-458

57. Heisler, L. E., Evangelou, A., Lew, A. M., Trachtenberg, J., Elsholtz, H. P., and Brown, T. J. (1997) Mol. Cell. Endocrinol. 126, 59-73

58. Lopez-Otin, C., and Diamandis, E. P. (1998) Endocrine Reviews 19, 365-396

59. Heinlein C A and Chang C Endocrine Reviews, in press.

60. Abraham, G. E. (1974) J. Clin. Endocrinol. Metab. 39, 340-346

61. Risch, H. A. (1998) J. Natl. Cancer Instit. 90, 1774-1786

62. Bunz, F., Dutriaux, A., Lengauer, C., Waldman, T., Zhou, S., Brown, J. P., Sedivy, J. M., Kinzler, K. W., and B., V. (1998) Science 282, 1497-1501

63. Brown, J. P., Wei, W., and Sedivy, J. M. (1997) Science 277, 831-834

64. Polyak, K., Waldman, T., He, T. C., Kinzler, K. W., and Vogelstein, B. (1996) Genes and Development 10, 1945-1952

65. Shaw, P. A., Rittenberg, P. V., and Brown, T. J. (2001) Gyn. Oncol. 80, 132-138

66. Silva, E. G., Tomos, C., Fritsche, H. A., El-Naggar, A., Gray, K., Ordonez, N. G., Luna, M., and Gershenson, D. (1997) Mod. Pathol. 10, 879-883

67. Engel, L. W., Young, N. A., Trala, T. S., Lippman, M. E., O'Brien, S. J., and Joyce, M. J. (1978) Cancer Research 38, 3352-3364

68. Nishizuka, Y., Sakakura, T., Tsujimura, T., and Matsumoto, K. (1972) Endocrinology 93, 786-792

69. Hild- Petito, S., West, N. B., Brenner, R. M., and Stouffer, R. L. (1991) Biollogy of Reproduction 44, 561-568

70. Horie, K., Takakura, K., Fujiwara, H., Suginami, H., Liao, S., and Mori, T. (1992) Human Reproduction 7, 184-190

71. Weil, S., Vendola, K., Zhou, J., and Bondy, C. A. (1999) J. Clin. Endocrinol. Metab. 84, 2951-2956

72. Tetsuka, M., and Hillier, S. G. (1996) Endocrinology 137, 4392-4397

73. Vendola, K, Zhou, J., Wang, J., Famuyiwa, O. A., Bievre, M., and Bondy, C. A. (1999) Biol. Reprod 61, 353-357

74. Zelinski-Wooten, M. B., and Stouffer, R. L. (1996) Trends Endocrinol. Metab. 7, 177-183

75. Koopman, P., Gubbay, J., Vivian, N., Goodfellow, P., and Lovell-Badge, R. (1991) Nature 351, 117-121

76. Ohno, S., and Christian, L., Attardi, B. (1973) Nature 243, 119-120

77. Lyon, M. F., and Glenister, P. H. (1980) Proc. R. Soc. Lond. B 208, 1-12

78. Lyon, M. F., and Glenister, P. H. (1974) Nature 274, 366-372

79. Schomberg, D. W., Couse, J. F., Mukherjee, A., Lubahn, D. B., Sar, M., Mayo, K. E., and Korach, K. S. (1999) Endocrinol., 2733-2744

80. Davis, S. R., McCloud, P. M., Strauss, B. J. G., and Burger, H. (1995) Maturitas 21, 227-236

81. Migliaccio, A., Castoria, G., Di Domenico, M., de Falco, A., Bilancio, A., Lombardi, M., Barone, M. V., Ametrano, D., Zannini, M. S., Abbondanza, C., and Auricchio, F. (2000) EMBO J. 19, 5406-5417

82. Panet-Raymand, V., Gottlieb, B., Beitel, L. K., Pinsky, L., and Trifiro, M. A. (2000) Mol. Cell. Endocrinol. 167, 139-150

83. Vanderschueren, D., van Herck, E., Suiker, A. M. H., Visser, W. J., Schot, L. P. C., Chung, K., Lucas, R. S., Einhorn, T. A., and Bouillon, R. (1993) J. Bone Miner. Res. 8, 801-809

84. Erben, R. G., Eberle, J., Stahr, K., and Goldberg, M. (2000) J. Bone Miner. Res. 15, 1085-1098

85. Uesugi, Y., Taguchi, O., Noumura, T., and Iguchi, T. (1992) Anat. Rec. 234, 541-548

86. Raisz, L. G. (1996) The Female Patient suppl., 11-18

87. Giguere, Y., Dewailly, E., Brisson, J., Ayotte, P., Laflamme, N., Demers, A., Forest, V. -I., Dodin, S., Robert, J., and Rousseau, F. (2001) Cancer Res. 61, 5869-5874

88. Xu, X., Wagner, K. -U., Larson, D., Weaver, Z., Li, C., Ried, T., Henninghausen, L., Wynshaw-Boris, A., and Deng, C. -X. (1999) Nat. Genet. 22, 37-43

89. Sandgren, E. P., Schroeder, J. A., Qui, T. H., Palmiter, R. D., Brinster, R. L., and Lee, D. C. (1995) Cancer Res. 55, 3915-3927

90. Chomczynski, P., and Sacchi, N. (1987) Analytical Biochemistry 162, 156-159

91. Gilliland, G., Perrin, S., Blanchard, K., and Bunn, H. F. (1990) Proc. Natl. Acad. Sci. USA 87, 2725-2729

92. Wagner, K. -U., Wall, R. J., St-Onge, L., Gruss, P., Wynshaw-Boris, A., Garrett, L., Li, M., Furth, P. A., and Hennighausen, L. (1997) Nucleic Acids Research 25, 4323-4330

93. Lydon, J. P., Ge, G., Kittrell, F. S., Medina, D., and O'Malley, B. W. (1999) Cancer Res. 59, 4276-4284

What is claimed is:

1. A composition comprising a cell, wherein the cell has a disrupted AR gene.

2. The composition of claim 1, wherein the cell is a breast cancer cell or breast cancer cell line.

3. The composition of claim 2, where in the breast cancer cell line is MCF-7, ZR-75-1, or T47-D.

4. The composition of claim 1, wherein the cell is an ovarian cancer cell or ovarian cancer cell line.

5. The composition of claim 4, where in the ovarian cancer cell line is OVCAR-3, ES-2, SKOV-3 or ovarian cancer cells.

6. The composition of claim 1, wherein the cell is an prostate cancer cell or prostate cancer cell line.

7. The composition of claim 1, wherein the cell line or cells is a prostate cancer cell line or prostate cancer cells, LNCaP cells or cell lines, or muscle cells or cell lines, bone cells or cell lines, brain cells or cell lines.

8. The composition of claim 1, wherein the cell is an embryonic stem cell, an embryonic germ cell, a breast cell, a breast cancer cell, an ovary cell, an ovary cancer cell, a prostate cell, a testis cell, a bone cell, a brain cell, a neural cell, or a muscle cell.

9. A transgenic mammal comprising the cell of claim 1.

10. A transgenic mammal comprising a disrupted AR gene.

11. The transgenic mammal wherein the disrupted AR gene encodes a protein that lacks a functional DNA binding domain.

12. The mammal of claim 10, wherein the mammal is a mouse.

13. The mammal of claim 10, wherein the disrupted AR gene lacks an exon 2 of the AR gene.

14. The mammal of claim 10, wherein the disrupted AR gene is produced by action of a recombinase.

15. The mammal of claim 14, wherein the recombinase is cre recombinase.

16. The mammal of claim 14, wherein the recombinase is under the control of an inducible promoter.

17. The mammal of claim 16, wherein the promoter is specific for breast, ovary, neural, bone, testis, liver, or prostate.

18. The mammal of claim 16, wherein the promoter is a WAP promoter or a ACTB promoter, a promoter specific for bone tissue.

19. A method of determining the effect of a steroid on AR comprising incubating the steroid with an AR disrupted cell line and assaying the effect of the steroid on the cell.

20. A method of evaluating treatment for cancer xenografts in ovarectomized mice comprising injecting a cell into the animal, wherein the cell has a disrupted AR loci.

21. The method of claim 20, wherein the cell is an MCF-7 cell, ZR-75-1 cell, T47-D cell, OVCAR-3 cell, ES-2 cell, or a SKOV-3 cell.

22. The method of claim 21, wherein the ovarectomized mice are nude mice.

23. An ovarectomized nude mouse comprising a xenograft wherein the xenograft comprises a cell injected into the mouse, wherein the cell has a disrupted AR loci.

24. The mouse of claim 23, wherein the cell comprises a breast cancer tissue line.

25. The mouse of claim 24, wherein in the breast cancer cell line is MCF-7, ZR-75-1, or T47-D.

26. The mouse of claim 23, wherein the cell comprises an ovarian cancer tissue line.

27. The mouse of claim 26, wherein in the ovarian cancer cell line is OVCAR-3, ES-2, or SKOV-3.

28. A method of evaluating tumor formation in an ARKO mouse, comprising injecting a cancer causing agent into the AKRO mouse.

29. The method of claim 28, wherein the cancer causing agent is MCF-7, ZR-75-1, T47-D, OVCAR-3, ES-2, or SKOV-3.
